(19) United States
(12) Patent Application Publication
(10) Pub. No.: US 2023/0282351 A1
(43) Pub. Date: Sep. 7, 2023

(54) DETECTION OF COGNITIVE IMPAIRMENT IN HUMAN BRAINS FROM IMAGES

(71) Applicant: Imperial College Innovations Limited, London (GB)

(72) Inventors: Eric Aboagye, London (GB); Marianna Inglese, London (GB)

(73) Assignee: Imperial College Innovations Limited, London (GB)

(21) Appl. No.: 17/798,921

(22) PCT Filed: Feb. 15, 2021
(86) PCT No.: PCT/GB2021/050365

§ 371 Date: Aug. 11, 2022

(30) Foreign Application Priority Data
Feb. 13, 2020 (GB) 2002020.2
Feb. 13, 2020 (GB) 2002021.0
Oct. 16, 2020 (GB) 2016471.1

(51) Int. Cl. G16H 50/20; G06T 7/11; G06T 7/00; G06T 7/155

(52) U.S. Cl. CPC G16H 50/20; G06T 7/11; G06T 7/0016; G06T 7/155; G06T 2207/20064; G06T 2207/10088; G06T 2207/20076; G06T 2207/30016

(57) ABSTRACT

A computer implemented method by which digital images of the human brain can be used to diagnose or to predict cognitive impairment, such as Alzheimer's disease and other forms of cognitive impairment such as so-called prodromal Alzheimer's disease. Methods of classifying or stratifying cohorts of human subjects such as for the purpose of clinical trials and/or to assess the impact of therapies are included. In some embodiments the images comprise T1 weighted MRI images.

### FIELD OF INVENTION

The present invention relates to methods and apparatus, and more particularly to computer implemented methods and computer apparatus, still more particularly, the invention relates to image processing for images of the human brain such as magnetic resonance images, and to methods and computer apparatus which use image processing for predicting and/or identifying cognitive impairment in the subject of such images.

### BACKGROUND

A variety of medical imaging techniques exist, and the spatial resolution achievable in these methods is increasingly accurate. Millimetre and even sub-millimetre resolution can often be achieved. The ability of these methods to distinguish between different types of tissue, and to identify structure within tissue, is also increasing, e.g. based on image contrast mechanisms of different types.

Accurate structural imaging can be performed by radiographic and tomographic techniques such as projection radiography and fluoroscopy, and X-ray computed tomography (CT), ultrasound techniques such as elastography, and magnetic resonance imaging (MRI). Other types of medical imaging are possible. For example, functional imaging techniques aim to provide contrast mechanisms which show underlying function. Techniques such as functional magnetic resonance imaging have been used for this purpose. Intuitively it would seem that the best way to investigate cognitive function is by use of functional neuroimaging techniques.

Another factor which militates against the use of structural images to predict cognitive changes or function is that whilst human anatomy might have some perceived norms there is a wide degree of variability between individuals which arise from both hereditary and environmental factors.

For example, the problem of how to map images of the brain of an individual subject onto a normalised anatomical model of the brain to facilitate comparisons between subjects and across populations is itself a non-trivial issue. In the context of such inherent variability, making predictions as to cognitive function based on structural data is fraught with potential error and difficulty.

A variety of cognitive disorders exist. Mild cognitive impairment (MCI) is a condition in which someone has minor problems with cognition—their mental abilities such as memory or thinking. In MCI these difficulties are worse than would normally be expected for a healthy person of their age. A person with MCI is more likely to go on to develop dementia.

Dementia is the name for a set of symptoms that includes memory loss and difficulties with thinking, problem-solving or language. Dementia develops when the brain is damaged by diseases, such as Alzheimer's disease. In Alzheimer's disease, among the areas often damaged first are the hippocampus. The amygdala and cortex are generally affected later than the hippocampus. The disease is however progressive, and may ultimately affect many regions of the brain. Damage to the hippocampus and amygdala may become measurable only after the symptoms of dementia are manifest.

Early diagnosis of MCI is a significant issue because once diagnosed steps can be taken to lessen the likelihood that a patient with MCI will go on to develop dementia.

### SUMMARY

Aspects and examples of the present disclosure aim to address technical problems related to those discussed above. In particular they may aim to provide a method of predicting or diagnosing cognitive impairment in a human subject based on images of the subject's brain.

An aspect of the disclosure provides a computer implemented method of predicting or diagnosing cognitive impairment in a human subject based on images of the subject's brain, the method comprising: identifying in the images, image regions corresponding to at least two of: the left cerebral cortex; the right cerebral cortex; the left hippocampus; and the left amygdala; and, determining for each of the identified image regions at least one image metric, wherein the or each at least one image metric of each image region provides a quantitative indication of structure in that image region; and determining an indicator based on the image metrics according to a predetermined method; obtaining reference data configured to indicate a cognitive impairment state using indicators determined according to the predetermined method; and predicting or diagnosing cognitive impairment in the subject based on comparing the indicator with the reference data.

Alternatively, different image regions may be used in this method instead. In one aspect, in place of the image regions listed above, the image regions may comprise the left choroid plexus; the right hippocampus; right cerebellar cortex; and the right inferior lateral ventricle.

In another aspect in place of the image regions listed above, the image regions may comprise the right middle temporal gyrus; the right rostral middle frontal; the right supramarginal; and the right temporal pole.

In an aspect there is provided a computer implemented method of distinguishing between human subjects the method comprising: determining, based on processing by the computer of digital image data obtained from the images, first image metrics providing a quantitative indication of structure in the left choroid plexus; the right hippocampus; right cerebellar cortex; and the right inferior lateral ventricle, determining a first indicator based on the first image metrics according to a first predetermined method; and distinguishing between Alzheimer's disease and non-Alzheimer's disease subjects based on the first indicator. Having identified subjects not having Alzheimer's disease in this way, the method may further comprise distinguishing between subjects having Alzheimer's disease and prodromal Alzheimer's disease determining, based on processing by the computer of digital image data obtained from the images, second image metrics providing a quantitative indication of structure in the right middle temporal gyrus; the right rostral middle frontal; the right supramarginal; and the right temporal pole determining a second indicator based on the second image metrics according to a second predetermined method thereby to distinguish between subjects having Alzheimer's disease and subjects having prodromal Alzheimer's disease.

In an aspect there is provided a method of distinguishing between Alzheimer's disease and non-Alzheimer's disease subjects. The method comprises a computer implemented method of predicting or diagnosing cognitive impairment in a human subject based on images of the subject's brain, the method comprising: determining, based on processing by the computer of digital image data obtained from the images, image metrics comprising at least one of: a complexity metric of an image region corresponding to the right middle temporal gyrus; an image texture metric of a image region corresponding to the right rostral middle frontal; an image texture metric in the image region corresponding to the right supramarginal; and an image intensity metric in the image region corresponding to the right temporal pole; the method further comprising: determining an indicator based on said image metrics according to a predetermined method; and predicting or diagnosing cognitive impairment state in the subject based on the indicator.

In an aspect there is provided a method of distinguishing between Alzheimer's disease and mild cognitive impairment (MCI) such as prodromal Alzheimer's disease. The method comprises a computer implemented method of predicting or diagnosing cognitive impairment in a human subject based on images of the subject's brain, the method comprising: determining, based on processing by the computer of digital image data obtained from the images, image metrics comprising at least one of an intensity measure of an image region corresponding to the left choroid plexus a morphological measure of the image region corresponding to the right hippocampus; an image texture in the image region corresponding to the right cerebellar cortex; and an image texture in the image region corresponding to the right inferior lateral ventricle; the method further comprising: determining an indicator based on said image metrics according to a predetermined method; and predicting or diagnosing cognitive impairment state in the subject based on the indicator.

The predetermined method of determining the indicator may comprise applying a weighting to each of the image metrics and combining the weighted image metrics, for example using a weighted sum. The combination may be linear. The first predetermined method may comprise a first set of weights, and a corresponding first set of image metrics such as any one of those described and claimed with reference to FIG. 13 and Table 13, below. The second predetermined method may comprise a second set of weights, and a corresponding second set of image metrics such as any one of those described and claimed with reference to FIG. 14 and Table 14, below.

One of the at least two image regions may be the left amygdala, and the at least one image metrics for the left amygdala may comprise a texture feature such as a non-uniformity measure. For example non-uniformity of a grey level size zone matrix, GLSZM.

At least one image metrics for the left amygdala may comprise a measure of correlation of the grey level co-occurrence matrix GLCM, such as an informational measure of correlation of the GLCM.

The image data upon which the image metrics described herein are based may be filtered in order to determine those image metrics, for example it may be high pass filtered, for example using a wavelet based filter, for example using a 3D high pass filter.

One of the at least two image regions may comprise the left hippocampus, and a run length non-uniformity, RLN, measure of a grey level run length matrix GLRLM may provide one of the image metrics of the image metrics of the left hippocampus.

One of the at least two image regions may comprise the left cerebral cortex, and the surface area to volume ratio may provide one of the image metrics of the left cerebral cortex. A measure of the degree to which the left cerebral cortex is spherical, for example its sphericity, may also provide one of the image metrics of the left cerebral cortex.

One of the at least one image metrics for the left cerebral cortex may be a fractal dimension maximum value, for example wherein the image region is high pass filtered in the first and third dimension and low pass filtered in the second dimension (HLH), for example using a wavelet based filter, for example using a 3D wavelet filter.

One of the at least two image regions may comprise the right hippocampus.

One of the at least one image metrics for the right hippocampus may comprise compactness, for example based on

$${F}_{morph . comp \text{.1}} = \frac{V}{{π}^{1 / 2} {A}^{3 / 2}} ,$$

wherein V is the volume of the right hippocampus image region and A is the surface area of the right hippocampus image region.

One of the at least two image regions may comprise the right cerebral cortex. One of the at least one image metrics for the right cerebral cortex may comprise the minimum value of the right cerebral cortex image region, for example filtered with a low pass filter, for example using a wavelet based filter, for example using a 3D low pass filter. One of the at least one image metrics for the right cerebral cortex may comprise a fractal dimension maximum value, for example wherein the image region is high pass filtered in the first and third dimension and low pass filtered in the second dimension (HLH), for example using a wavelet based filter, for example using a 3D wavelet filter.

One of the at least two image regions may comprise the left inferior lateral ventricle. One of the at least one image metrics for the left inferior lateral ventricle may comprise a measure of correlation of the grey level co-occurrence matrix GLCM, such as an informational measure of correlation of the GLCM. The image data upon which the GLCM is based may be low pass filtered in the first and third dimension and high pass filtered in the second dimension (LHL), for example using a wavelet based filter, for example using a 3D wavelet filter.

An aspect of the disclosure provides a computer implemented method of predicting or diagnosing cognitive impairment in a human subject based on images of the subject's brain, the method comprising: identifying in the images, image regions corresponding to the left amygdala, the left inferior lateral ventricle, the left hippocampus, and the right hippocampus; determining for each of the identified image regions at least one image metric, wherein the or each at least one image metric of each image region provides a quantitative indication of structure in that image region; and determining an indicator based on the image metrics according to a predetermined method; obtaining reference data configured to indicate a cognitive impairment state using indicators determined according to the predetermined method; and predicting or diagnosing cognitive impairment in the subject based on comparing the indicator with the reference data.

The image metric for the left inferior lateral ventricle may comprise a correlation measure of the grey level co-occurrence matrix, GLCM, (such as information based correlation). The image region may be filtered, for example using an LHL wavelet filter.

The image metric for the left hippocampus may comprise a run length non-uniformity measure of the grey level run length matrix, GLRLM.

The image metric for the left amygdala may comprise a grey level non-uniformity measure of the grey level size zone matrix, GLSZM.

The image metric for the right hippocampus may comprise compactness.

An aspect of the disclosure provides a computer implemented method of predicting or diagnosing cognitive impairment in a human subject based on images of the subject's brain, the method comprising: identifying, in the images, image regions corresponding to sub-regions of the cortex comprising: the banks of the superior temporal sulcus; the lateral occipital lobe; the entorhinal cortex; and the pars orbitalis; determining for each of the identified image regions a corresponding at least one image metric, wherein the at least one image metric comprises at least one of: (i) a measure of image texture; (ii) a measure of image intensity in the image region, such as its central tendency; and (iii) a morphological measure of the image region determining an indicator based on the image metrics according to a predetermined method; obtaining reference data configured to indicate a cognitive impairment state using indicators determined according to the predetermined method; and predicting or diagnosing cognitive impairment in the subject based on comparing the indicator with the reference data.

The sub-region of the cortex in the banks of the superior temporal sulcus may comprise the right banks of the superior temporal sulcus. The sub-region of the cortex in the lateral occipital lobe may comprise the left lateral occipital lobe. The sub-region of the cortex in the entorhinal cortex may comprise the left entorhinal cortex. The sub-region of the cortex in the pars orbitalis may comprise the left pars orbitalis.

The at least one image metric of the entorhinal cortex, such as the left entorhinal cortex, may comprise the measure of image intensity such as at least one of: (a) a measure of its central tendency, for example the mean; and (b) a measure of its spread such as its maximum and/or minimum.

The at least one image metric of the left entorhinal cortex may comprise the measure of image texture, such as a metric of correlation in the grey level co-occurrence matrix which may be used in combination with a small zone emphasis of the grey level size zone matrix.

The at least one image metric of the left lateral occipital lobe may comprise the metric of image texture, such as a neighbourhood grey tone based feature—for example a measure of complexity in the neighbourhood grey tone difference matrix, NGTDM.

The at least one image metric of the banks of the right superior temporal sulcus may comprise at least one of: (a) the measure of image intensity, for example the measure of central tendency; and (b) the measure of image texture such as a measure based on the grey level co-occurrence matrix such as the sum variance, e.g.

$${F}_{cm . sum . var} = {\sum }_{k = 2}^{2 {N}_{g}} {( k - µ )}^{2} {p}_{i + j , k}$$

An aspect provides a computer implemented method of predicting or diagnosing cognitive impairment in a human subject based on images of the subject's brain, the method comprising: identifying, in the images, image regions corresponding to sub-regions of the cortex comprising: the left inferior temporal lobe, the left entorhinal cortex the left superior parietal lobe, the left middle temporal lobe, and the right superior parietal lobe, determining for each of the identified image regions a corresponding at least one image metric, wherein the at least one image metric comprises at least one of: (i) a measure of image texture; (ii) a measure of image intensity in the image region, such as its central tendency; and (iii) a morphological measure of the image region determining an indicator based on the image metrics according to a predetermined method; obtaining reference data configured to indicate a cognitive impairment state using indicators determined according to the predetermined method; and predicting or diagnosing cognitive impairment in the subject based on comparing the indicator with the reference data.

An aspect of the disclosure provides a computer implemented method of predicting or diagnosing cognitive impairment in a human subject based on images of the subject's brain, the method comprising: identifying in the images, image regions corresponding to the left amygdala and at least one of: the left cerebral cortex; the right cerebral cortex; and the left hippocampus; and, determining for each of the identified image regions at least one image metric, wherein the or each at least one image metric of each image region provides a quantitative indication of structure in that image region; and determining an indicator based on the image metrics according to a predetermined method;

obtaining reference data configured to indicate a cognitive impairment state using indicators determined according to the predetermined method; and predicting or diagnosing cognitive impairment in the subject based on comparing the indicator with the reference data.

One of the at least one image metrics for the left amygdala may comprise compactness. One of the at least one image metrics for the left amygdala may comprise a run length non-uniformity measure. For example it may be based on a grey level run length matrix, GLRLM, for example wherein it comprises at least one of GLRLM run length non uniformity and GLRLM grey level non-uniformity.

One of the at least one image regions may be the left hippocampus and one of the at least one image metrics for the left hippocampus may be based on compactness and/or a texture strength, for example texture strength based on neighbourhood grey tone difference.

The image regions used in the methods described herein may comprise subcortical regions such as: Entorhinal cortex, Fusiform gyrus, Banks of the superior temporal sulcus, Inferior parietal lobe, isthmus cingulate, Medial orbitofrontal, Middle temporal, Parahippocampal, Rostral middlefrontal, Superior parietal and so forth.

One such aspect of the disclosure comprises a computer Implemented method of predicting or diagnosing cognitive impairment in a human subject based on images of the subject's brain, the method comprising: identifying in the images, an image region corresponding to the fusiform gyrus, determining for the identified image region at least one image metric, wherein the at least one image metric comprises at least one of: (i) a texture feature indicating the prevalence of small zones having low grey level; (ii) a measure of central tendency, such as the mode; and (iii) a texture feature indicating the prevalence of short run lengths having low grey level thereby to provide a quantitative indication of structure in that image region; determining an indicator based on the image metrics according to a predetermined method; obtaining reference data configured to indicate a cognitive impairment state using indicators determined according to the predetermined method; and predicting or diagnosing cognitive impairment in the subject based on comparing the indicator with the reference data.

This method has been tested on a test population comprising control subjects and subjects having mild cognitive disfunction, and has been found to be able to distinguish between the two. It may also be able to distinguish AD subjects from control subjects.

One such aspect of the disclosure provides a computer Implemented method of predicting or diagnosing cognitive impairment in a human subject based on images of the subject's brain, the method comprising: identifying in the images, an image region corresponding to the entorhinal cortex, determining for the identified image region at least one image metric, wherein the at least one image metric comprises at least one of: (i) a morphological feature indicating the extent to which the region is spherical, for example spherical disproportion; (ii) a texture feature indicating the prevalence of small zones having high grey level; (iii) the minimum fractal dimension; and (iv) a measure of texture strength, such as a the texture strength of the neighbourhood grey tone difference matrix (NGTDM); thereby to provide a quantitative indication of structure in that image region; and determining an indicator based on the image metrics according to a predetermined method; obtaining reference data configured to indicate a cognitive impairment state using indicators determined according to the predetermined method; and predicting or diagnosing cognitive impairment in the subject based on comparing the indicator with the reference data.

This method has been tested on a test population comprising control subjects and subjects having Alzheimer's Disease, and has been found to be able to distinguish between the two. It may also be able to distinguish MCI subjects from control subjects. This method may be further refined by identifying in the images, an image region corresponding to the fusiform gyrus, determining for the image region corresponding to the fusiform gyrus at least one of the following image metrics: (i) a texture feature indicating the prevalence of small zones having low grey level; (ii) a measure of central tendency, such as the mode; and

(iii) a texture feature indicating the prevalence of short run lengths having low grey level thereby to provide the quantitative indication of structure in the image region corresponding to the fusiform gyrus.

The texture feature indicating the prevalence of small zones having low grey level may comprise the grey level size zone (GLSZM) small zone low grey level emphasis.

$${F}_{szm . szlge} = \frac{1}{{N}_{s}} {\sum }_{i = 1}^{{N}_{g}} {\sum }_{j = 1}^{{N}_{z}} \frac{{s}_{ij}}{{i}^{2} {j}^{2}}$$

The texture feature indicating the prevalence of short run lengths having low grey level comprises the GLRLM Short Run Low Gray Level Emphasis (SRLGLE)

$${F}_{rlm . srlge} = \frac{1}{{N}_{s}} {\sum }_{i = 1}^{{N}_{g}} {\sum }_{j = 1}^{{N}_{r}} \frac{{r}_{ij}}{{i}^{2} {j}^{2}}$$

Embodiments of the disclosure provide computer program products configured to program a programmable processor to perform any of the methods described and/or claimed herein. Embodiments also provide tangible non transitory computer readable media storing such computer program products, for example in the form of computer readable instructions.

Embodiments of the disclosure also provide computer apparatus configured to perform any of the methods described and/or claimed herein.

Embodiments of the disclosure provide methods of diagnosing cognitive impairment, for example MCI or dementia, for example Alzheimer's disease. These and other embodiments may enable early treatment to be provided, e.g. before the impairment can be detected by traditional diagnostic techniques.

Embodiments of the present disclosure may provide methods of selecting subjects for participation in clinical trials. For example, such embodiments may comprise selecting patients based on the indicators defined herein.

Embodiments also provide methods of classifying subjects and/or identifying cohorts of patients in such trials, for example to permit intra-cohort and inter-cohort comparison amongst or between cohorts of patients identified based on the indicators defined herein.

Some embodiments provide methods of treating cognitive impairment, the methods comprising determining an indicator of cognitive impairment according to any one or more of the methods described and/or claimed herein, and selecting a therapy, such as a medicament, dosage regimen, or course of treatment based on said indicator.

The images may comprise images having contrast able to distinguish between tissue types in the human brain, and may have sufficient resolution to permit them to resolve structures in the human brain such as cortex, gyri and sulci of the grey matter, white matter structures such as cortico-cortical fibres, bundles of such fibres and so forth. The image contrast mechanism and/or the resolution may also be sufficient to enable aggregates of grey matter within cerebral white matter to be resolved. For example, the images may be capable of resolving sub-structures which make up one or more of the following anatomical features of the brain:
Left Cerebral White Matter
Left Inferior Lateral Ventricle
Left Cerebral Cortex
Right Cerebral Cortex
Left Hippocampus
Left Amygdala
Left choroid plexus
Brain Stem
White matter (WM) hypointensities, which may comprise white matter lesions
Left Pallidum
Corpus Callosum Posterior
Left Inferior Lateral Ventricle
Right Hippocampus.

Any imaging technique capable of resolving such sub-structures may be used. For example, imaging methods able to distinguish water, water protein mixtures, and fat may be used. The images may have a resolution of at least 3 mm resolution or finer, for example at least 2 mm resolution, for example at least 1 mm. The images may have a resolution of better than 1 mm, for example 0.6 mm. The resolution may be isotropic. The images may be converted to 1 mm resolution by pre-processing steps such as those performed by FreeSurfer to enable image segmentation (e.g. they may be downsampled, and or smoothed e.g. with antialiasing filters, and so forth).

In an aspect there is provided a computer implemented method of predicting or diagnosing cognitive impairment in a human subject based on images of the subject's brain, the method comprising: identifying, in the images, image regions corresponding to sub-regions of the cortex comprising: the fusiform gyrus, the entorhinal cortex the temporal pole, the transverse temporal cortex, and the insula, determining for each of the identified image regions a corresponding at least one image metric, wherein the at least one image metric comprises at least one of: (i) a measure of image texture (i) a measure of image intensity in the image region, such as its central tendency; and (iii) a morphological measure of the image region determining an indicator based on the image metrics according to a predetermined method; obtaining reference data configured to indicate a cognitive impairment state using indicators determined according to the predetermined method; and predicting or diagnosing cognitive impairment in the subject based on comparing the indicator with the reference data.

The at least one image metric of the entorhinal cortex may comprise a measure of its central tendency, for example the mean and/or the morphological measure. The image region corresponding to the entorhinal cortex may correspond primarily to the left entorhinal cortex or primarily to the right entorhinal cortex, and different image metrics may be used in the left entorhinal cortex as compared to those used in the right entorhinal cortex. The morphological measure in the entorhinal cortex may comprise an indication of shape, such as sphericity for example spherical distortion.

The at least one image metric in the entorhinal cortex may comprise the measure of image texture, such as a measure indicating the degree of heterogeneity in grey level. The measure of image texture may comprise a texture feature based on grey level zone size, such as a feature indicating the prevalence of small zone size. The measure of image texture may comprise a texture feature based on the grey level run length, such as a feature indicating the prevalence of short runs.

The image region corresponding to the fusiform gyrus may correspond primarily to the left fusiform gyrus e.g. in preference to the right (e.g. only to the left). In this region the metric of the left fusiform gyrus may comprise: (a) a first order statistic indicating grey level intensity, such as a measure of central tendency or minimum, and/or (b) the measure of image texture, such as a measure indicating the degree of heterogeneity in grey level. This measure of image texture may comprise a texture feature based on the grey level run length, such as a feature indicating the prevalence of short runs, for example those having low grey level.

The image region corresponding to the temporal pole may correspond primarily to the right temporal pole, for example in preference to the left (e.g. only to the right). In this context image metric of the temporal pole may comprise a first order statistic indicating grey level intensity, such as a measure of central tendency.

The image region corresponding to the transverse temporal cortex may correspond primarily to the right transverse temporal cortex, for example in preference to the left (e.g. only to the right). In this context, the image metric of the transverse temporal cortex comprises a first order statistic indicating grey level intensity, such as a measure or central tendency or distribution

The image region corresponding to the insula may correspond primarily to the right insula, for example in preference to the left (e.g. only to the right). In this context, the image metric of the insula may comprise the measure of image texture, such as a measure indicating the degree of heterogeneity in grey level. The measure of image texture may comprise a texture feature based on the grey level run length, such as a feature indicating the prevalence of short runs.

The image region corresponding to the entorhinal cortex may correspond primarily to the right entorhinal cortex, for example in preference to the left (e.g. only to the right). In this context, the at least one image metric of the entorhinal cortex may comprise a measure of image intensity such as a measure of its spread such as its maximum and/or minimum. The at least one image metric of the entorhinal cortex may comprise the measure of image texture, such as a metric of correlation in the grey level co-occurrence matrix this metric may be used in combination with a metric of the grey level size zone matrix, such as a metric indicating the prevalence of small zones.

The image regions may comprise a sub region of the cortex corresponding to the lateral occipital lobe, which may correspond primarily to the left lateral occipital lobe, for example in preference to the right (e.g. only to the left). The at least one image metric of the left lateral occipital lobe may comprise the metric of image texture, such as a neighbourhood grey tone based feature—for example a measure of complexity in the neighbourhood grey tone difference matrix, NGTDM.

The image regions may comprise a region which corresponds to the banks of the superior temporal sulcus, and may correspond primarily to the right banks of the superior temporal sulcus, for example in preference to the left (e.g. only to the right). The at least one image metric of the right banks of the superior temporal sulcus comprises at least one of: (a) the measure of image intensity, for example the measure of central tendency; and (b) the measure of image texture such as a measure based on the grey level co-occurrence matrix.

The image regions may comprise a sub region of the cortex corresponding to the caudal middle frontal cortex which may correspond primarily to the left caudal middle frontal cortex, for example in preference to the right (e.g. only to the left). In this context, the at least one image metric of the right caudal middle frontal cortex may comprise the metric of image texture, such as a neighbourhood grey tone based feature—for example a measure of complexity in the neighbourhood grey tone difference matrix, NGTDM.

The image regions may comprise a sub region of the cortex corresponding to the isthmus cingulate, which may correspond primarily to the left isthmus cingulate, for example in preference to the right (e.g. only to the left). The at least one image metric of the left isthmus cingulate may comprise a measure of image intensity, such as a measure of central tendency—e.g. the mean.

The image regions may comprise a sub region of the cortex corresponding to the rostral middle frontal cortex, which may correspond primarily to the right rostral middle frontal cortex, for example in preference to the left (e.g. only to the right). The at least one image metric of the right rostral middle frontal may comprise the measure of image intensity.

The methods described with reference to the above described aspect and the embodiments of that aspect may comprise age normalising the images prior to identifying the image regions and determining said image metrics.

The image region corresponding to the entorhinal cortex may correspond primarily to the left entorhinal cortex. The at least one image metric of the left entorhinal cortex may comprise at least one of: (a) a metric of the grey level size zone matrix; and (b) a metric of the neighbourhood grey tone difference matrix.

The image regions may comprise a sub region of the cortex corresponding primarily to the left inferior parietal lobe, and the image metric of the left inferior parietal lobe may comprise a metric of image texture such as a metric of the grey level run length matrix.

The image regions may comprise a sub region of the cortex corresponding to the lateral occipital lobe and the image metric comprises a measure of image texture such as a measure of the fractal dimension for example a standard deviation of the fractal dimension.

The image regions may comprise a sub region of the cortex corresponding to the pars triangularis. For example, corresponding primarily to the left (e.g. in age normalised images) and alternatively to the right in non-age normalised data. The image metric of the pars triangularis may comprise a measure of image intensity such as a measure of central tendency.

The image regions may comprise a sub region of the cortex corresponding to the rostral middle frontal cortex, for example primarily to the left rostral middle frontal cortex. The at least one image metric of the rostral middle frontal cortex may comprise the measure of image texture, such as a measure based on the grey level size zone matrix, such as a metric indicating the variance of the zone sizes.

The image regions may comprise a sub region of the cortex corresponding to the superior temporal lobe (e.g. primarily to the left superior temporal lobe) and the image metrics of the left superior temporal lobe may comprise the measure of image texture, for example a metric based on at least one of: (a) the grey level size zone matrix; and (b) the grey level co-occurrence matrix.

The image regions may comprise a sub region of the cortex corresponding to the supramarginal cortex (e.g. primarily to the left supramarginal cortex) and the image metric of the left supramarginal cortex may comprise the measure of image texture, for example a measure based on the grey level size zone matrix.

The image metric of the temporal pole (e.g. primarily to the left temporal pole) may comprise a measure of image texture based on fractal dimension such as a minimum fractal dimension.

The image region corresponding to the insula may correspond primarily to the right insula in which case the image metric in the right insula may comprise a measure of image intensity such as a measure of central tendency, for example the mode.

A further aspect of the disclosure may provide a computer Implemented method of predicting or diagnosing cognitive impairment in a human subject based on images of the subject's brain. This aspect may find particular application in differentiating between subjects having Alzheimer's disease and control subjects. The method comprises: identifying, in the images, image regions corresponding to sub-regions of the cortex comprising: the inferior temporal lobe, the entorhinal cortex the superior parietal lobe, the middle temporal lobe, and the superior parietal lobe, determining for each of the identified image regions a corresponding at least one image metric, wherein the at least one image metric comprises at least one of: (i) a measure of image texture; (ii) a measure of image intensity in the image region, such as its central tendency; and (iii) a morphological measure of the image region determining an indicator based on the image metrics according to a predetermined method; obtaining reference data configured to indicate a cognitive impairment state using indicators determined according to the predetermined method; and predicting or diagnosing cognitive impairment in the subject based on comparing the indicator with the reference data.

In this aspect, the image metric in the inferior temporal lobe may correspond primarily to the left inferior temporal lobe in preference to the right (e.g. only to the left) and may comprise a measure of image intensity, such as a measure of central tendency. The at least one image metric in the entorhinal cortex may correspond primarily to the left entorhinal cortex and may comprise a measure of image texture such as a metric of the neighbourhood grey tone difference matrix, for example a contrast metric of the NGTDM.

The at least one image metric in the superior parietal lobe may correspond primarily to the left superior parietal lobe and may comprise a measure of image texture, such as a metric of the grey level co-occurrence matrix, such as autocorrelation of the grey level co-occurrence matrix.

The at least one image metric in the superior parietal lobe may correspond primarily to the right superior parietal lobe and may comprise a measure of image intensity such as the minimum.

The at least one image metric in the middle temporal lobe correspond primarily to the left middle temporal lobe and may comprises a measure of image intensity such as the maximum.

Embodiments of the present disclosure provide a computer implemented method of differentiating between human subjects having at least mild cognitive impairment, MCI, and control subjects, the method comprising obtaining images of each subject's brain, and performing any one or more of the methods described or claimed herein to differentiate between the subjects.

Embodiments of the present disclosure also provide a method of differentiating between human subjects having Alzheimer's Disease, AD, and control subjects, the method comprising obtaining images of each subject's brain, and performing any one or more of the methods described or claimed herein to differentiate between the subjects.

The methods described herein may comprise age normalising the image data prior to determining said at least one image metric. Normalising may comprise: (a) obtaining, for each voxel, j, a vector of intensity values wherein each intensity value comprises the intensity of the voxel, j, voxel in one of a corresponding plurality of images each obtained from a control subject (CN). The vector of intensity values at the voxel j may be denoted as xⱼ^{CN} (b) obtaining a vector of age values, each age value identifying the age of a corresponding one of the control subjects a^{CN} so that each element of the vector of age values corresponds to an element of the vector of intensity values; (c) determining parameters of age related effects in the voxel, j, based on the vector of age values and the vector of intensity values and a model of said effects; and (d) using said model and said parameters to normalise intensity values in the voxel j in images of test subjects based on the age of said test subject.

The model of age related effects may comprise a linear model, such as a straight line or other linear model. Determining the parameters may comprise fitting said model to the vector of intensity values. For example, fitting may comprise least squares fitting of the model

xⱼ^{NC}=αⱼa^{NC}+αⱼ₀.

to determine the parameters αⱼ and αⱼ₀.

The model, and these parameters, can then be applies to images of test subjects (e.g. the MCI and AD groups) to remove the age effects of each voxel separately. For example, by estimating the age related effect in each voxel j for each subject based on the model and the parameters, and subtracting these effects from the corresponding intensity in said voxel, j.

For example, for the MCI group:

xⱼ^{MCIar}=xⱼ^{MCII}−αⱼa^{MCI}+αⱼ₀.

Embodiments of the present disclosure also provide method of classifying subjects for a clinical study, the method comprising obtaining images of each subject's brain, and performing any one or more of the methods described or claimed herein to differentiate between the subjects.

Embodiments of the disclosure provide a computer program product, such as a tangible non-transitory computer readable medium storing program instructions configured to program a processor to perform the method of any preceding claim.

It will be appreciated in the context of the present disclosure that the images described herein, whether relating to control subjects or to test subjects may be coregistered with each other and/or with a reference image of the human brain. Such reference images may comprise an anatomical standard.

Magnetic resonance images having T1 contrast, such as T1 weighted MRI, may be particularly useful in the methods described herein. Other imaging methods having contrast mechanisms able to distinguish between different types of human brain tissue may also be used to provide the images upon which the methods described herein operate.

### BRIEF DESCRIPTION OF THE DRAWINGS

Embodiments of the disclosure will now be described in detail, by way of example only, with reference to the accompanying drawings, in which:

FIG. 1 shows an apparatus configured to predict or diagnose cognitive impairment;

FIG. 2 shows a plot of the ROC analysis corresponding to Table 1 and Table 3;

FIG. 3 shows a plot of the ROC analysis corresponding to Table 2 and Table 4;

FIG. 4 shows the results of an ROC analysis for the comparative tests set out in Table 7;

FIG. 5 shows the results of an ROC analysis for the comparative tests set out in Table 8;

FIG. 6 shows a set of overlays of particular image features in sub-regions of the cortex;

FIG. 7 shows further overlays obtained from particular image features in sub regions of the cortex;

FIG. 8 shows further overlays obtained from particular image features in sub regions of the cortex;

FIG. 9 shows the results of an ROC analysis for the feature set defined in Table 10 an indicates the results of a comparative analysis;

FIG. 9 shows the results of an ROC analysis for the feature set defined in Table 11 and indicates the results of a comparative analysis;

FIG. 10 shows the results of an ROC analysis for the feature set defined in Table 12 and indicates the results of a comparative analysis;

FIG. 11 shows the results of an ROC analysis for the feature set defined in Table 11 and indicates the results of a comparative analysis;

FIG. 12 shows the results of an ROC analysis for the feature set defined in Table 12 and indicates the results of a comparative analysis;

FIG. 13 shows the results of an ROC analysis for the feature set defined in Table 13 and indicates the results of a comparative analysis; and

FIG. 14 shows the results of an ROC analysis for the feature set defined in Table 14 and indicates the results of a comparative analysis.

### DETAILED DESCRIPTION

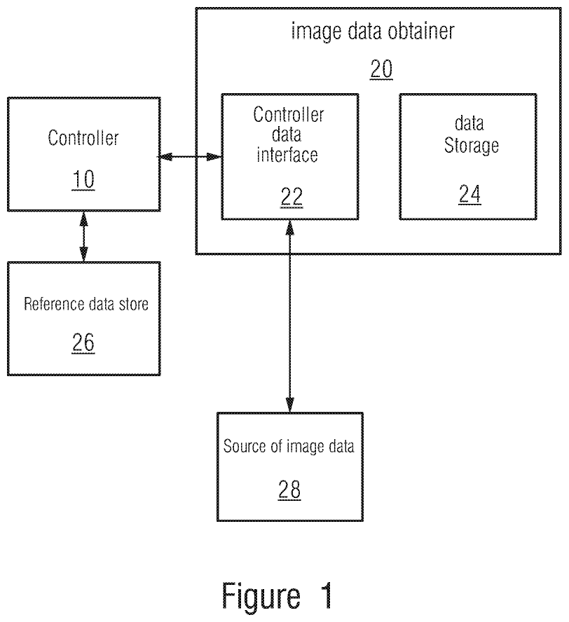

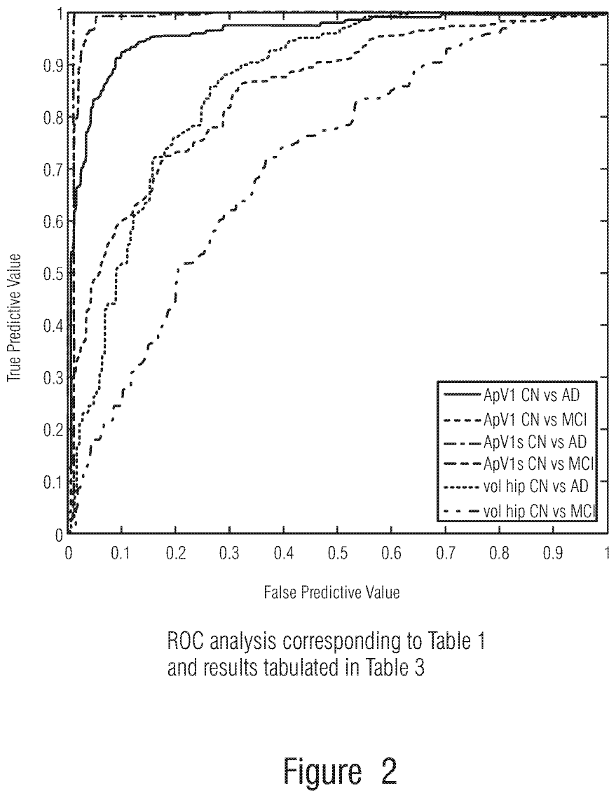

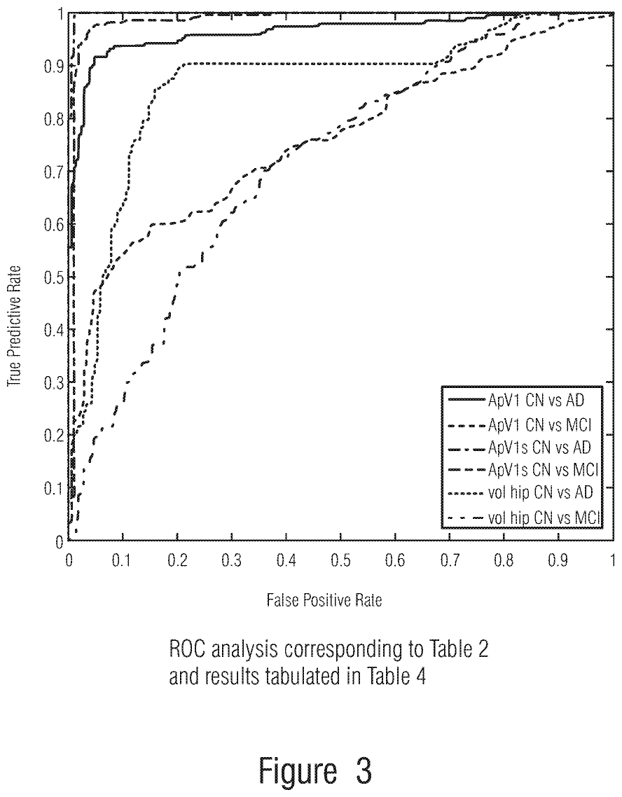

A computer implemented method of predicting or diagnosing cognitive impairment in a human subject will now be described with reference to the apparatus shown in FIG. 1.

In overview, this apparatus segments the images to identify in those images, image regions corresponding to selected anatomical features in the brain. It then determines an image metric or metrics for each anatomical features (segmented image region). Each of these image metrics provides a quantitative indication of structure in that image region, which are combined according to a predetermined method to determine an indicator. The apparatus then obtains reference data against which the indicator is compared to predict or diagnose cognitive impairment in the subject.

The apparatus illustrated in FIG. 1 comprises a subject image data obtainer 20, a reference data store 26, and a controller 10. It may also be coupled to a source of image data 28.

The subject image data obtainer 20 may comprise a data interface 22 for communicating data with the controller 10 and/or with the source of image data 28. It may also comprise volatile and/or non-volatile data storage 24, such as a cache, connected to the data interface 22. The subject image data obtainer 20 may be connected (e.g. via the interface 22) to communicate with one or both of:
an imaging system such as a CT scanner, or an MRI scanner or other imaging system capable of acquiring appropriate images of the human brain; or
a store of appropriate images of the human brain.

Other sources of image data (such as network connections to local and/or wide area networks may also be used). Images of subjects can thus be provided to the controller 10 from a variety of sources.

The controller 10 may comprise a general purpose processor or similar processing logic, which is configured to segment images of the human brain according to a brain atlas model such as that defined in the freesurfer utilities which are available from https://surfer.nmr.mgh.harvard.edu/fswiki/CorticalParcellation. It will be appreciated in the context of the present disclosure that such utilities may assign neuroanatomical labels to each location on a cortical surface model based on probabilistic information estimated from a manually labelled training set (e.g. that which is made using FreeSurfer). Such procedures may incorporate both geometric information derived from the cortical model, and neuroanatomical convention, as found in the training set. The atlases used may comprise:
gyral based atlases such as the ‘Desikan-Killiany’ cortical atlas described in “An automated labeling system for subdividing the human cerebral cortex on MRI scans into gyral based regions of interest” Neuroimage 31 (2006) 968-980, by RS Desikan et al.
atlases based on parcellation schemes that divide the cortex into gyral and sulcal regions such as the ‘Destrieux’ cortical atlas described in “Automatic parcellation of human cortical gyri and sulci using standard anatomical nomenclature” NeuroImage 53, Issue 1, 15 Oct. 2010, Pages 1-15, Destrieux et al.
the DKT40 atlas https://mindboggle.info/data.html or other similar atlases. Whatever atlas is used, the controller may be configured to identify, in image data having appropriate contrast and resolution, some or all of the following structures:
Left Cerebral White Matter
Left Inferior Lateral Ventricle
Left Cerebral Cortex
Right Cerebral Cortex
Left Hippocampus
Left Amygdala
Left choroid plexus
Brain Stem
White matter (WM) hypointensities, which may comprise white matter lesions
Left Pallidum
Corpus Callosum Posterior
Left Inferior Lateral Ventricle
Right Hippocampus

The controller may also be configured to segment, from the images regions of interest (ROI) corresponding to the structures which it identifies. This too may be done using approaches such as those defined in the FreeSurfer package or other equivalent packages.

The controller also comprises image processing functionality arranged to determine some or all of the features defined below. For example, the controller may be configured to determine for a given ROI:
First order statistics such as measures of central tendency, measures of spread, skewness and kurtosis;
Morphological features;
Grey Level Size Zone Matrix features;
Grey Level Co-occurrence Matrix (GLCM) features;
Neighbourhood grey tone difference based features;
Grey Level Run Length Matrix (GLRLM) features; and
Fractal dimension features.

Examples of such features and the way in which they may be determined by the controller are explained below. It will be appreciated in the context of the present disclosure that other equivalent and/or comparable image features may be used. In addition, the formulae given for the features listed are intended only to serve as an illustrative description of the way in which they can be provided. Typically, such metrics operate on digital data which may be encoded as a discrete grey level in each voxel of an ROI. It will be appreciated in the context of the present disclosure that the notations set out below, using indices and summations are intended to be implemented by the controller stepping through the intensity values (e.g. digital grey level data) stored in the pixels of the images (or ROI's of those images). The indices mentioned in the mathematical formulae below may thus connote the indices of stepwise computational methods, such as may be implemented using a counter incremented to step through the relevant set of digital data. Other methods, such as vector methods, may also be used.

### Morphological Features

Morphological features, such as the surface area, A, and volume, V, of a region identified in an image may be determined based on voxel representations of that volume. Mesh based representations of the outer surface of such a volume may also be used to determine the surface area and volume of the region, for example a marching cubes algorithm may be used.

One morphological feature which may be used is the degree to which a region is spherical. One measure of this feature is sphericity, which may be defined by F_{morph.sphericity}

$${F}_{morph . sphericity} = \frac{{( 36 π {V}^{2} )}^{1 / 3}}{A}$$

wherein V is the volume of the image region concerned, and A is its surface area. Other measures include:
compactness, which may be based on

$${F}_{morph . comp \text{.1}} = \frac{V}{{π}^{1 / 2} {A}^{3 / 2}}$$

(so called compactness 1), or

$${F}_{morph . comp \text{.2}} = 36 π \frac{{V}^{2}}{{A}^{3}}$$

(so called compactness 2)
asphericity, which may be based on the ratio A³/V² and spherical disproportion, which may be based on the ratio A/V^{2/3}.

### Grey Level Size Zone Matrix Features

The grey level size zone matrix (GLSZM) counts the number of groups (or zones) of linked voxels. Voxels are linked if the neighbouring voxel has an identical discretised grey level. Whether a voxel classifies as a neighbour depends on its connectedness. In a 3D approach to texture analysis all of the 26 neighbouring voxels in a 3D rectilinear grid are considered. In the 2D the neighbouring 8 voxels in the same 2D image are considered.

Let M be the Ng×Nz grey level size zone matrix, where Ng is the number of discretised grey levels present in the ROI intensity mask and Nz the maximum zone size of any group of linked voxels. Element sᵢⱼ of M is then the number of zones with discretised grey level i and size j. Furthermore, let Nᵥ be the number of voxels in the intensity mask and

Nₛ=Σᵢ₌₁^{N}^{_{g}}Nⱼ₌₁^{N}^{_{z}}sᵢⱼ

be the total number of zones. Marginal sums can likewise be defined. The number of zones with discretised grey level i, regardless of size is s_{i.}

s_{i.}=Σⱼ₌₁^{N}^{_{z}}sᵢⱼ

Likewise, the number of zones with size j, regardless of grey level is s·j

s_{.j}=Σᵢ₌₁^{N}^{_{g}}sᵢⱼ

A grey level non-uniformity measure can then be defined which assesses the distribution of zone counts over the grey values. The feature value is low when zone counts are equally distributed along grey levels. One example of a grey level size zone matrix based grey level non-uniformity measure, denoted F_{szm.glnu}, comprises:

$${F}_{szm . glnu} = \frac{1}{{N}_{s}} {\sum }_{i = 1}^{{N}_{g}} {s}_{i .}^{j}$$

The controller may be configured to determine an image metric which gives emphasis to the prevalence of large zones. One example of such a measure, which may be based on the grey level size zone matrix and indicate the presence of large zones, is the large zone emphasis of the grey level size zone matrix. This may be denoted F_{szm.lze} and comprises:

$${F}_{szm . lze} = \frac{1}{{N}_{s}} {\sum }_{j = 1}^{{N}_{z}} {j}^{2} {s}_{. j}$$

The controller may be configured to determine an image metric indicating the variance in zone counts over the different zone sizes (the GLSZM variance)

$${F}_{szm . zs . var} = {\sum }_{i = 1}^{{N}_{g}} {\sum }_{j = 1}^{{N}_{z}} {( j - μ )}^{2} {p}_{ij}$$

In which pᵢⱼ=sᵢⱼ/Nₛ and μ=Σᵢ₌₁^{N}^{_{g}}Σⱼ₌₁^{N}^{_{z}}jpᵢⱼ

### Grey Level Co-Occurrence Matrix (GLCM) Features

The grey level co-occurrence matrix (GLCM) is a matrix that expresses how combinations of discretised intensities (grey levels) of neighbouring pixels, or voxels in a 3D volume, are distributed along one of the image directions. Generally, the neighbourhood for GLCM is a 26-connected neighbourhood in 3D and a 8-connected neighbourhood in 2D. Thus, in 3D there are 13 unique direction vectors within the neighbourhood for Chebyshev distance δ=1, i.e. (0, 0, 1), (0, 1, 0), (1, 0, 0), (0, 1, 1), (0, 1, −1), (1, 0, 1), (1, 0, −1), (1, 1, 0), (1, −1, 0), (1, 1, 1), (1, 1, −1), (1, −1, 1) and (1, −1, −1), whereas in 2D the direction vectors are (1, 0, 0), (1, 1, 0), (0, 1, 0) and (−1, 1, 0).

A GLCM is calculated for each direction vector, as follows. Mₘ is the N_{g}×N_{g} grey level co-occurrence matrix, with N_{g} the number of discretised grey levels present in the region of interest (ROI) intensity mask, and m the particular direction vector.

Element (i, j) of the GLCM contains the frequency at which combinations of discretised grey levels i and j occur in neighbouring voxels along direction m₊=m and along direction m₋=−m. Then, Mₘ=Mₘ₊+Mₘ₋=Mₘ₊+M^{T}ₘ₊. As a consequence the GLCM matrix Mₘ is symmetric.

A probability distribution for grey level co-occurrences, Pₘ, is obtainable by normalising Mₘ by the sum of its elements. Each element pᵢⱼ of Pm is then the joint probability of grey levels i and j occurring in neighbouring voxels along direction m.

The row marginal probability pᵢ is

p_{i.}=Σⱼ₌₁^{N}^{_{g}}pᵢⱼ

and the column marginal probability is

p_{.j}=Σᵢ₌₁^{N}^{_{g}}pᵢⱼ

as Pₘ is by definition symmetric, p_{i.}=p_{.j}.

Measures of correlation of the grey level co-occurrence matrix GLCM can be defined such as

$${F}_{cm . corr} = \frac{1}{{σ}_{i .} {σ}_{. j}} ( - {μ}_{i .} {μ}_{. j} + {\sum }_{i = 1}^{{N}_{g}} {\sum }_{j = 1}^{{N}_{s}} {ijp}_{ij} )$$

in which μ_{i.} and σ_{i.} are the mean and standard deviation of row marginal probability p_{i.}, respectively. Likewise, μ_{.j} and σ_{.j} are the mean and standard deviation of the column marginal probability p_{.j}, respectively. An informational measure of correlation of the grey level co-occurrence matrix GLCM, F_{cm.info.corr.2}:

$${F}_{cm . info . corr \text{.2}} = \sqrt{1 - exp ( - 2 ( {HXY}_{2} - HXY ) )}$$

$$wherein ,$$

$$HXY = - {\sum }_{i = 1}^{{N}_{g}} {\sum }_{j = 1}^{{N}_{g}} {p}_{ij} {log}_{2} {p}_{ij} ,$$

$${HXY}_{2} = - {\sum }_{i = 1}^{{N}_{g}} {\sum }_{i = 1}^{{N}_{s}} {p}_{i .} {p}_{. j} {log}_{2} ( {p}_{i .} {p}_{. j} )$$

pᵢⱼ is the joint probability of grey levels i and j occurring in neighbouring voxels along a direction in which the GLCM is defined;

p_{i.} is the row marginal probability of the GLCM, and

p_{.j} is the column marginal probability of the GLCM.

Other measures of correlation of the GLCM may be used.

The controller may be configured to determine the above and other measures of correlation. In addition, the controller may be configured to determine an auto correlation of the GLCM based on:

$${F}_{cm . auto . corr} = {\sum }_{i = 1}^{{N}_{g}} {\sum }_{j = 1}^{{N}_{g}} {ijp}_{ij}$$

The GLCM difference variance may be computed as the difference variance for the diagonal probabilities thus:

$${F}_{cm . diff . var} = {\sum }_{k = 0}^{{N}_{g} - 1} {( k - μ )}^{2} {p}_{i - j . k}$$

The sum average for the GLCM is:

$${F}_{cm . sum . avg} = {\sum }_{k = 2}^{2 {N}_{g}} {kp}_{i + j . k}$$

The sum variance for the GLCM is defined as:

$${F}_{cm . sum . var} = {\sum }_{k = 2}^{2 {N}_{g}} {( k - μ )}^{2} {p}_{i + j . k}$$

Where μ is equal to the value of the sum average for the GLCM.

The controller may be configured to determine an entropy measure of the GLCM, such as a sum entropy:

$${F}_{cm . sum . entr} = - {\sum }_{k = 2}^{2 {N}_{g}} {{p}_{i}}_{+ j . k} {log}_{2} {p}_{i + j . k}$$

It can thus be seen that a variety of metrics of image texture may be obtained from the GLCM. Another example is the inverse variance:

$${F}_{cm . inv . var} = 2 {\sum }_{i = 1}^{{N}_{g}} {\sum }_{j > 1}^{{N}_{g}} \frac{{p}_{ij}}{{( i - j )}^{2}}$$

The controller may also be configured to determine other measures derived from the GLCM—such as cluster based measures of texture. One such measure is the cluster tendency

$${F}_{cm . clust . tend} = {\sum }_{i = 1}^{{N}_{g}} {\sum }_{j = 1}^{{N}_{g}} {( i + j - {μ}_{i .} - μ {.}_{j} )}^{2} {p}_{ij}$$

in which μᵢ is the mean row marginal probability, and μ_{.j} is the mean column marginal probability. As noted above, these parameters may be computed as

μ_{i.}=Σᵢ₌₁^{N}^{_{g}}i p_{i.} and μ_{.j}=Σⱼ₌₁^{N}^{_{g}}j p_{.j}

Another such cluster based measure is the so called GLCM cluster shade, which may be computed as

$${F}_{cm . clust . shade} = {\sum }_{i = 1}^{{N}_{g}} {\sum }_{j = 1}^{{N}_{g}} {( i + j - {μ}_{i .} - {μ}_{. j} )}^{3} {p}_{ij}$$

Another such cluster based measure is the so called GLCM cluster prominence, which may be computed as

$${F}_{cm . clust . prom} = {\sum }_{i = 1}^{{N}_{g}} {\sum }_{j = 1}^{{N}_{g}} {( i + j - {μ}_{i .} - {μ}_{. j} )}^{4} {p}_{ij}$$

### Neighbourhood Grey Tone Difference Based Features

The controller may also be configured to determine neighbourhood grey tone difference matrix (NGTDM). This contains the sum of grey level differences of pixels/voxels with discretised grey level i and the average discretised grey level of neighbouring pixels/voxels within a Chebyshev distance δ.

The average grey level within a neighbourhood centred at (kₓ, k_{y}, k_{z}), but excluding (kₓ, k_{y}, k_{z}) itself is:

$${\overline{X}}_{k} = \frac{1}{W} {\sum }_{{m}_{x} m - δ}^{δ} {\sum }_{{m}_{y} m - δ}^{δ} {\sum }_{{m}_{x} m - δ}^{δ} {X}_{d} ( {k}_{x} + {m}_{x} , {k}_{y} + {m}_{y} , {k}_{z} + {m}_{z} )$$

$$( {m}_{x} , {m}_{y} , {m}_{z} ) \neq ( 0 , 0 , 0 )$$

where X_{d,k} is the discretised grey level of a voxel at position kₓ, k_{y}, k_{z}), for a 3D neighbourhood W=(2δ+1)3−1 is the size of the neighbourhood. For a 2D neighbourhood W=(2δ+1)2−1, and averages are not calculated between different slices.

Neighbourhood grey tone difference sᵢ for discretised grey level i is:

$${s}_{i} = {\sum }_{i = 1}^{{N}_{v}} \vert i - {\overline{X}}_{k} \vert [ {X}_{d} ( k ) = i and {W}_{k} \neq 0 ]$$

where, Wₖ is neighbourhood size for the voxel (kₓ, k_{y}, k_{z}) and Nᵥ is equal to the number of voxels in the neighbourhood that are part of the ROI mask

$${W}_{k} = {\sum }_{m , m - δ}^{δ} {\sum }_{{m}_{8} m - δ}^{δ} {\sum }_{m , m - δ}^{δ} ⌈ m \neq 0 and k + m in ROI ⌉$$

where [ . . . ] is an Iverson bracket, which is 1 if the conditions within it are true and zero otherwise.

In NGTDM grey level probabilities pᵢ are defined, thus pᵢ=nᵢ/N_{v,c}. N_{v,c} is total number of voxels that have at least one neighbour. If all voxels have at least one neighbour N_{v,c}=Nᵥ

The controller may be configured to determine a texture strength based on the NGTDM. One example of such a texture strength comprises:

$${F}_{ngi \cdot strength} = \frac{{\sum }_{{i}_{1} = 1}^{{N}_{g}} {\sum }_{{i}_{2} = 1}^{{N}_{g}} ( {p}_{{i}_{1}} + {p}_{{i}_{2}} ) {( {i}_{1} - {i}_{2} )}^{2}}{{\sum }_{i = 1}^{{N}_{g}} {s}_{i}} ,$$

$${p}_{{i}_{1}} \neq 0 and {p}_{{i}_{2}} \neq 0$$

Where N_{g} is the number of discretised grey levels in the ROI intensity mask.

The controller may be configured to determine a contrast based on the NGTDM, which may be based on depends on the dynamic range of the grey levels and the spatial frequency of intensity changes in said grey levels. One example of such a contrast comprises:

$${F}_{ngt \cdot contrast} = ( \frac{1}{{N}_{g , p} ( {N}_{g , p} - 1 )} {\sum }_{{i}_{1} = 1}^{{N}_{g}} {\sum }_{{i}_{2} = 1}^{{N}_{g}} {p}_{{i}_{1}} {{p}_{{i}_{2}} ( {i}_{1} - {i}_{2} )}^{2} )$$

$$( \frac{1}{{N}_{v , c}} {\sum }_{i = 1}^{{N}_{g}} {s}_{i} )$$

Grey level probabilities pᵢ₁ and pᵢ₂ are copies of pᵢ with different iterators, i.e. pᵢ₁=pᵢ₂ for i₁=i₂. The first term considers the grey level dynamic range, whereas the second term is a measure for intensity changes within the volume. If N_{g,p}=1, F_{ngt.contrast}=0.

The controller may also be configured to determine a measure of busyness, for example based on the prevalence of large changes in grey levels between neighbouring voxels. One such metric may be defined

$${F}_{ngt \cdot busyness} = \frac{{\sum }_{i = 1}^{{N}_{g}} {p}_{i} {s}_{i}}{{\sum }_{{i}_{1} = 1}^{{N}_{g}} {\sum }_{{i}_{2} = 1}^{{N}_{g}} \vert {i}_{1} {p}_{{i}_{1}} - {i}_{2} {p}_{{i}_{2}} \vert} ,$$

$${p}_{{i}_{1}} \neq 0 and {p}_{{i}_{2}} \neq 0$$

The controller may also be configured to determine a measure of complexity such as an image metric indicating the prevalence of complex textures in which rapid changes in grey levels are common. One example of such a metric is texture complexity, or NGTDM complexity, which may be defined:

$${F}_{ntg \cdot complexity} = \frac{1}{{N}_{v , c}} {\sum }_{{i}_{1} = 1}^{{N}_{g}} {\sum }_{{i}_{2} = 1}^{{N}_{g}} \vert {i}_{1} - {i}_{2} \vert \frac{{p}_{{i}_{1}} {s}_{{i}_{1}} + {p}_{{i}_{2}} {s}_{{i}_{2}}}{{p}_{{i}_{1}} + {p}_{{i}_{2}}} ,$$

$${p}_{{i}_{1}} \neq 0 and {p}_{{i}_{2}} \neq 0$$

The controller may be configured to provide a metric of coarseness based on the fact that grey level differences in coarse textures are generally small due to large-scale patterns. Such measures may be determined by summing differences to give an indication of the level of the spatial rate of change in intensity (coarseness). One measure of NGTDM coarseness may be defined as:

$${F}_{ngt \cdot coarseness} = \frac{1}{{\sum }_{i = 1}^{{N}_{g}} {p}_{i} {s}_{i}}$$

Where N_{g}, sᵢ and pᵢ are defined as above.

### Grey Level Run Length Matrix (GLRLM) Features

Another way to define texture features is to use the grey level run length matrix (GLRLM). A run length is defined as the length of a consecutive sequence of pixels or voxels with the same grey level along direction m. The GLRLM then contains the occurrences of runs with length j for a discretised grey level i.

Mₘ is an Ng×Nr grey level run length matrix, where Ng is the number of discretised grey levels present in the ROI intensity mask and Nr is the maximum possible run length along direction m. Matrix element rᵢⱼ of the GLRLM is the occurrence of grey level i with run length j. If Nᵥ is the total number of voxels in the ROI, and Ns is the sum over all elements in Mₘ

In the context of GLRLM measures, r_{i.} is the marginal sum of the runs over run lengths j for grey value i.

r_{i.}=Σⱼ₌₁^{N}^{ᵣ}rᵢⱼ.

Similarly, in GLRLM measures r_{.j} is the marginal sum of the runs over the grey values i for run length j.

r_{.j}=Σᵢ₌₁^{N}^{_{g}}rᵢⱼ.

The controller may be configured to determine grey level non-uniformity, e.g. based on r_{i.} the marginal sum of the runs over run lengths j for grey value i, viz.

$${F}_{rlm \cdot glnu} = \frac{1}{{N}_{s}} {\sum }_{i = 1}^{{N}_{g}} {r}_{i}^{2}$$

where Nₛ is the sum over all elements in Mₘ in the GLRLM.

The controller may also be configured to determine run length non-uniformity based on r_{.j} the marginal sum of the runs over the grey values i for run length j. For example, this may comprise an image metric whose value is low when runs are equally distributed along run lengths, e.g.

$${F}_{rlm \cdot rlnu} = \frac{1}{{N}_{s}} {\sum }_{j = 1}^{{N}_{g}} {r}_{j}^{2}$$

The controller may also be configured to determine measures of run percentage such as the fraction of the number of realised runs and the maximum number of potential runs. Strongly linear or highly uniform ROI volumes may produce low run percentages. One example of run percentage may be defined:

$${F}_{rlm \cdot r \cdot perc} = \frac{{N}_{s}}{{N}_{v}} .$$

Any feature of any one of the examples disclosed herein may be combined with any selected features of any of the other examples described herein. For example, features of methods may be implemented in suitably configured hardware, and the configuration of the specific hardware described herein may be employed in methods implemented using other hardware.

### Fractal Dimension Features

A fractal dimension of a region may be based on a statistical index of complexity comparing how detail in a pattern, such as the boundary of an image region changes with the scale at which it is measured. It may also be based on the space-filling capacity of such a pattern. Fractal dimensions considers the space filling properties of the images, and may be defined based on methods such as those defined in “A multifractal approach to space-filling recovery for PET quantification.” Med Phys. 2014 November; 41(11):112505. doi: 10.1118/1.4898122. Willaime J M, Aboagye E O, Tsoumpas C, Turkheimer F E.

### Spatial Filtering

In addition to the image metrics defined above, the controller may also be configured to apply filters such as high pass and/or low pass filters to the image data (e.g. prior to calculation of the image metrics). These filters may be wavelet based. It will be appreciated in the context of the present disclosure that three-dimensional wavelets can be constructed as separable products of 1-D wavelets by successively applying a 1-D analyzing wavelet in three spatial directions (x, y, z). The volume F (x, y, z) is firstly filtered along the x-dimension, resulting in a low-pass image L(x, y, z) and a high-pass image H(x, y, z). Both L and H are then filtered along the y-dimension, resulting in four decomposed sub-volumes: LL, LH, HL and HH. Then each of these four subvolumes are filtered along the z-dimension, resulting in eight sub-volumes: LLL, LLH, LHL, LHH, HLL, HLH, HHL and HHH.

In 1D dimension, the continuous wavelet transform is defined as the convolution of x(t) with a wavelet function, W(t), shifted in time by a translation parameter b and a dilation parameter a:

$${X}_{W} ( a , b ) = \frac{1}{\sqrt{a}} {\int }_{- ∞}^{+ ∞} W \frac{t - b}{a} x ( t ) dt$$

The discrete form of the wavelet transform is based upon the discretization of parameters (a, b) on the time-scale plane corresponding to a discrete set of continuous basis functions. This can be achieved defining:

$${W}_{j , k} ( t ) = \frac{1}{\sqrt{{a}_{j}}} W \frac{( t - {b}_{k} )}{{a}_{j}}$$

For aⱼ=a₀^{j} and bₖ=kb₀ a₀^{j} where j, k∈Z, a₀>1, b₀≠0 where j controls the dilation and k controls the translation. Two popular choices for the discrete wavelet parameters a₀ and b₀ are 2 and 1 respectively, a configuration that is known as the dyadic grid arrangement resulting in:

$${W}_{j , k} ( t ) = {a}_{0}^{- \frac{j}{2}} W ( {a}_{0}^{- j} t - k {b}_{0} ) = {2}^{- \frac{j}{2}} W ( {2}^{- j} t - k )$$

Wavelet analysis is simply the process of decomposing a signal into shifted and scaled versions of a mother (initial) wavelet. An important property of wavelet analysis is perfect reconstruction, which is the process of reassembling a decomposed signal or image into its original form without loss of information. For decomposition and reconstruction the scaling function Φⱼₖ(t) and the wavelet Wⱼₖ(t) are used in the form:

$${Φ}_{j k} ( t ) = {2}^{- \frac{j}{2}} {Φ}_{0} ( {2}^{- j} t - k )$$

$${W}_{j k} ( t ) = {2}^{- \frac{j}{2}} {Ψ}_{0} ( {2}^{- j} t - k )$$

where m stands for dilation or compression and k is the translation index. Every basis function W is orthogonal to every basis function Φ.

The one-dimensional wavelet transform of a discrete-time signal x(n) (n=0, 1, . . . , N) is performed by convolving signal x(n) with both a half-band low-pass filter L and high-pass filter H and downsampling by two.

$$c ( n ) = {\sum }_{n - 0}^{L - 1} {h}_{0} ( k ) x ( n - k )$$

$$d ( n ) = {\sum }_{n - 0}^{L - 1} {h}_{1} ( k ) x ( n - k )$$

where c(n) represent the approximation coefficients forn=0, 1, 2 . . . ,N−1 and d(n) are the detail coefficients, h0 and h1, are coefficients of the discrete-time filters L and H respectively where c(n) represent the approximation coefficients for n=0, 1, 2 . . . , N−1 and d(n) are the detail coefficients, h₀ and h₁, are coefficients of the discrete-time filters L and H respectively.

{h₀(n)}ₙ₋₀^{L-1}=(h₀(0),h₀(1), . . . ,h₀(L−1))

{h₁(n)}ₙ₋₀^{L-1}=(h₁(0),h₁(1), . . . ,h₁(L−1))

resulting in the separable, sub-band process.

It will be appreciated in the context of the present disclosure that whilst wavelet based filters may have particular advantages, other types of spatial filters may be used.

### IMPLEMENTATION

By applying the image processing steps outlined above, the controller is configured first to segment the image data for the subject to identify regions of the image data (anatomical neuroanatomical regions of interest, ROIs). It will be appreciated in the context of a 3D image (such as one made up of a set of slices) that an ROI may comprise a 3D volume, e.g. a cluster of voxels spanning more than one slice of a volumetric image.

For each ROI, the controller determines a selected one or more of the features mentioned above, e.g.
First order statistics such as measures of central tendency, measures of spread, skewness and kurtosis;
Morphological features;
Grey Level Size Zone Matrix features;
Grey Level Co-occurrence Matrix (GLCM) features;
Neighbourhood grey tone difference based features;
Grey Level Run Length Matrix (GLRLM) features; and
Fractal dimension features.

The controller may store a list of weightings which define the weighting to be given to that feature (image metric) in combining the feature score from each region to provide an indicator. Two examples of such lists of weights have been defined for predicting or diagnosing cognitive impairment in human subjects. The first of these is defined in Table 1, below. The second is defined in Table 2. These two lists have been found to have excellent prediction accuracy, but prediction accuracy which is sufficient to make reliable diagnosis may be provided by other embodiments such as those described and claimed elsewhere herein.

TABLE 1

| | Region | Feature | Weight |
|---|---|---|---|
| a | Left Cerebral | median, and ROI may be | 0.3119 |
| | White Matter | filtered, e.g. using | |
| | | an LHH filter | |
| b | Left Cerebral Cortex | Compactness 1 | −0.4431 |
| c | Left Cerebral Cortex | Mode | 1.2220 |
| d | Left Inferior | A correlation measure of the | −1.4074 |
| | Lateral Ventricle | GLCM (e.g. information | |
| | | based correlation) | |
| e | Left Hippocampus | Compactness 2 | −1.0632 |
| f | Left Hippocampus | NGTDB Based measure of | 1.7741 |
| | | texture strength, and ROI | |
| | | may be filtered, e.g. | |
| | | using an HLL filter | |
| g | Left Amygdala | Compactness 1 | −2.5940 |
| h | Left Amygdala | GLRLM run length non | −0.0486 |
| | | uniformity, and ROI may | |
| | | be filtered, e.g. using | |
| | | an HLL filter | |
| i | Left Amygdala | GLRLM grey level non | −0.8351 |
| | | uniformity, and ROI may | |
| | | be filtered, e.g. using | |
| | | an HHH filter | |
| j | Right Cerebral Cortex | mode | 1.70381 |

It may be advantageous to age normalise image data before applying the feature analysis defined in Table 1.

TABLE 2

| | Region | Feature | Weight |
|---|---|---|---|
| A | Left Cerebral Cortex | Shape to volume ratio | 0.7590 |
| B | Left Cerebral Cortex | Sphericity | −0.4395 |
| C | Left Cerebral Cortex | Maximum fractal dimension, and | −0.4646 |
| | | ROI may be filtered, e.g. | |
| | | using an HLH filter. | |
| D | Left Inferior | Standard deviation, and ROI | −0.0156 |
| | Lateral Ventricle | may be filtered, e.g. using | |
| | | an LHL filter | |
| E | Left Inferior | A correlation measure of the | −1.2023 |
| | Lateral Ventricle | GLCM (e.g. information based | |
| | | correlation), and ROI may be | |
| | | filtered, e.g. using an | |
| | | LHL filter | |
| F | Left Pallidum | GLSZM large zone emphasis | −0.6226 |
| G | Brain Stem | Maximum fractal dimension, and | 0.1561 |
| | | ROI may be filtered, e.g. | |
| | | using an HHL filter. | |
| H | Left Hippocampus | Minimum, and ROI may be | −0.5028 |
| | | filtered, e.g. using an | |
| | | LLL filter. | |
| I | Left Hippocampus | GLRLM run length non uniformity | −1.4729 |
| J | Left Amygdala | GLSZM grey level non uniformity | −1.4162 |
| K | Left Amygdala | correlation measure of | 0.6181 |
| | | the GLCM (e.g. information | |
| | | based correlation), and | |
| | | ROI may be filtered, | |
| | | e.g. using an HHH filter | |
| L | Left choroid plexus | Maximum fractal dimension | −0.0894 |
| M | Right Cerebral Cortex | Minimum, and ROI may be | −0.9432 |
| | | filtered, e.g. using an | |
| | | LLL filter | |
| N | Right Cerebral Cortex | Maximum, and ROI may be | −0.5396 |
| | | filtered, e.g. using an | |
| | | HLH filter | |
| O | Right Hippocampus | Compactness | −1.5872 |
| P | WM hypointensities | GLCM correlation, and ROI | −0.3731 |
| | | may be filtered, e.g. | |
| | | using an HLL filter | |
| Q | Corpus Callosum | GLCM Maximum probability, | −0.2500 |
| | Posterior | and ROI may be filtered, | |
| | | e.g. using an LHL filter. | |
| R | Corpus Callosum | GLCM Entropy, and ROI | 0.5234 |
| | Posterior | may be filtered, e.g. | |
| | | using an LHL filter | |

The controller may apply either (a) the combination of weightings listed in Table 1, or (b) the combination of weightings listed in Table 2 to scale each feature's value in the corresponding ROI before combining the scaled feature values (e.g. by summing them) to provide the indicator. The controller then compares this indicator value with reference data thereby to determine a cognitive impairment diagnosis for the indicator.

The reference data may be predetermined using the same set of regions, features, and weights as is used to determine the indicator, but based on image data from subjects having a known cognitive impairment diagnosis. The reference data may thus comprise reference value (or range of values) associated with each population on image data from subjects having a known cognitive impairment diagnosis (e.g. control (no impairment), MCI, AD, etc.). The reference data may also be correlated with cognitive testing scores, which may enable a cognitive testing score to be estimated or predicted based on the image data.

The indicators defined by the combinations of regions, features and weights defined in Table 1 are listed in Table 3. The rows shown in Table 3 indicate the outcome of an ROC analysis.

In Table 3 the first of the two columns under the heading APV1 (CNvsAD) indicates the application to a trial population of Alzheimer's disease (AD) subjects and control subjects (CN), and the accuracy of the method defined in table 1 in discriminating AD from CN. The second of the two columns under the heading APV1 (CNvsMCI) indicates the application to a trial population of mild cognitive impairment (MCI) subjects and control subjects (CN), and the accuracy of the method defined in Table 1 in discriminating MCI from CN. The columns under the heading Vol Hippocampus indicate the accuracy of discrimination based on the so-called “gold standard” measure provided using the volume of the hippocampus. Compared to the gold standard, our method shows higher AUC, specificity, sensitivity, accuracy, negative and positive predictive values, likelihood ratios and diagnostic odds ratios in both the discriminations between CN/MCI and CN/AD. Table 4 provides the same ROC analysis for indicators defined by the combinations of regions, features and weights defined in Table 2.

Table 5 shows the same ROC analysis for indicators defined by the combinations of regions, features and weights defined in Table 1 to a different cohort. Table 6 shows the same ROC analysis for indicators defined by the combinations of regions, features and weights defined in Table 2 to a different cohort. The data used to establish tables 5 and 6 provided by OASIS, namely OASIS-3: Principal Investigators: T. Benzinger, D. Marcus, J. Morris; NIH P50AG00561, P30NS09857781, P01AG026276, P01AG003991, R01AG043434, UL1TR000448, R01EB009352. Any AV-45 doses were provided by Avid Radiopharmaceuticals, a wholly owned subsidiary of Eli Lilly.

It will be appreciated in the context of the present disclosure that the weights listed herein need not be given the accuracy quoted here. It is believed that weightings which provide comparable relative contributions of at least two of the more heavily weighted contributions may provide reliable prediction/diagnosis.

TABLE 3

| | |
|---|---|
| ApV1 | Vol Hippocampus |

| | CNvsAD | CNvsMCI | CNvsAD | CNvsMCI |
|---|---|---|---|---|
| AUC | 0.9578 | 0.8477 | 0.8600 | 0.7144 |
| Threshold | −2.1161 | −2.4288 | 0.3793 | 0.4399 |
| Specificity | 0.9027 | 0.8240 | 0.8564 | 0.7222 |
| Sensitivity | 0.9214 | 0.7281 | 0.7382 | 0.6313 |
| Accuracy | 0.9115 | 0.7759 | 0.8009 | 0.6766 |
| NPV | 0.9285 | 0.7510 | 0.7872 | 0.6610 |
| PPV | 0.8934 | 0.8061 | 0.8197 | 0.6954 |
| LR+ | 9.4779 | 4.1387 | 5.1437 | 2.2728 |
| LR− | 0.0869 | 0.3299 | 0.2444 | 0.5104 |
| YI | 0.8242 | 0.5521 | 0.6505 | 0.3535 |
| DOR | 108.9524 | 12.5441 | 18.0225 | 4.4525 |

TABLE 4

| | |
|---|---|
| ApV2 | Vol Hippocampus |

| | CNvsAD | CNvsMCI | CNvsAD | CNvsMCI |
|---|---|---|---|---|
| AUC | 0.9642 | 0.7596 | 0.8792 | 0.7146 |
| Threshold | −4.8105 | −5.2179 | 0.3573 | 0.4399 |
| Specificity | 0.9537 | 0.8518 | 0.8981 | 0.7175 |
| Sensitivity | 0.9162 | 0.6036 | 0.8315 | 0.6313 |
| Accuracy | 0.9361 | 0.7274 | 0.8669 | 0.6743 |
| NPV | 0.9279 | 0.6814 | 0.8584 | 0.6595 |
| PPV | 0.9459 | 0.8036 | 0.8777 | 0.6919 |
| LR+ | 19.7905 | 4.0748 | 8.1679 | 2.2355 |
| LR− | 0.0878 | 0.4652 | 0.1875 | 0.5137 |
| YI | 0.8699 | 0.4555 | 0.7296 | 0.3489 |
| DOR | 225.3125 | 8.7587 | 43.5397 | 4.3514 |

TABLE 5

| | ApV1-OAS | | Volume hip | |
|---|---|---|---|---|
| | CNvsAD | CNvsUND | CNvsAD | CNvsUND |
| AUC | 0.7409 | 0.7050 | 0.7774 | 0.7328 |
| Threshold | −2.0260 | −2.1677 | 0.4137 | 0.5011 |
| Specificity | 0.7467 | 0.7012 | 0.6667 | 0.7308 |
| Sensitivity | 0.6666 | 0.6923 | 0.8766 | 0.6688 |
| Accuracy | 0.7326 | 0.7000 | 0.8396 | 0.6778 |
| NPV | 0.9126 | 0.9310 | 0.5366 | 0.2714 |
| PPV | 0.3606 | 0.2812 | 0.9247 | 0.9364 |
| LR+ | 2.2402 | 2.2792 | 7.1037 | 2.2065 |
| LR− | 0.3798 | 0.4314 | 0.3802 | 0.4532 |
| YI | 0.4134 | 0.3936 | 0.5433 | 0.3996 |
| DOR | 5.8974 | 5.2826 | 14.2098 | 5.4818 |

TABLE 6

| | |
|---|---|
| ApV1-OAS | Volume hip |

| | CNvsAD | CNvsMCI | CNvsAD | CNvsMCI |
|---|---|---|---|---|
| AUC | 0.7884 | 0.6726 | 0.7357 | 0.6655 |
| Threshold | −4.9773 | −5.5365 | 0.4054 | 0.5461 |
| Specificity | 0.7878 | 0.6484 | 0.6000 | 0.7429 |
| Sensitivity | 0.6857 | 0.6857 | 0.8606 | 0.5636 |
| Accuracy | 0.7700 | 0.6550 | 0.8150 | 0.5950 |
| NPV | 0.9219 | 0.9067 | 0.4773 | 0.2653 |
| PPV | 0.4067 | 0.2926 | 0.9103 | 0.9118 |
| LR+ | 2.5068 | 2.0633 | 4.3042 | 1.7023 |
| LR− | 0.3093 | 0.5126 | 0.2323 | 0.5874 |
| YI | 0.4735 | 0.3341 | 0.4606 | 0.3065 |
| DOR | 8.1038 | 4.0250 | 9.2604 | 3.7259 |

It can be seen that in all cases, and in both cohorts of subjects, the ability of the method described herein to distinguish control subjects from those with MCI or AD matches or exceeds measurements based on the volume of the hippocampus.

### The Use of Reduced Feature Sets

It is believed that the weightings having the highest absolute values may contribute most strongly to the predictive power of the indicators defined by the lists of tuples defined in Table 1 and Table 2. Therefore not all of the features and regions need be used in order to predict cognitive impairment. Particularly advantageous combinations of features and regions include those described and claimed elsewhere herein.

To determine the extent to which reduced feature sets may be useful in predicting cognitive impairment two comparative studies were conducted. In the first comparative study the predictive power of the full feature sets listed above in Table 1 was compared with the predictive power of incomplete versions of that same set. In summary—it was determined that the proposal set out in the preceding paragraph is correct, and that methods of predicting or diagnosing cognitive impairment based only on the more strongly weighted image metrics and image regions provide effective predictive power.

In Tables 7 and 8 listed below the tuples of feature and region listed in Tables 1 and 2 are referred to using row labels. This labelling relates to the image regions and image metrics identified in those rows of Table 1 and Table 2 being used to determine an indicator for comparison with reference data, thereby to predict or diagnose cognitive impairment in the subject in the manner described and claimed herein.

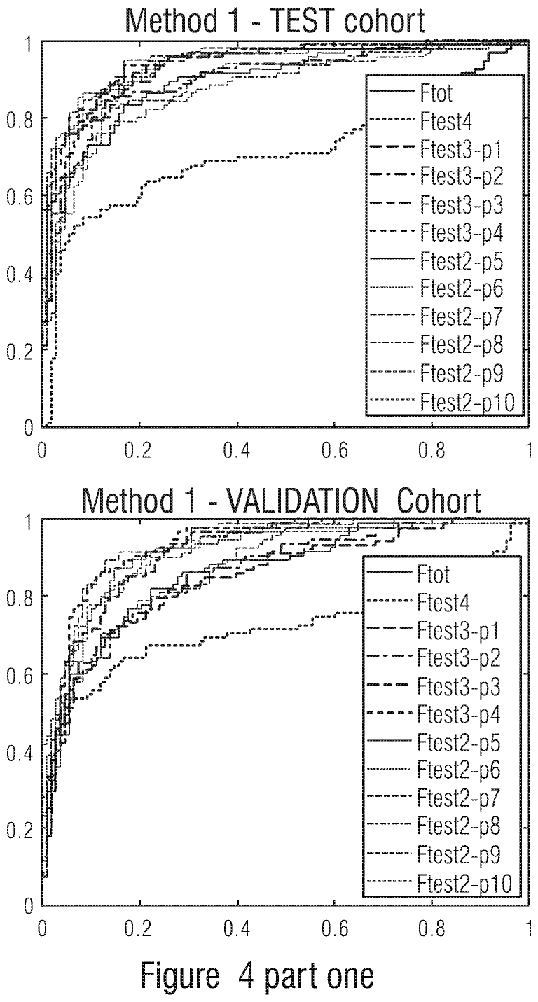

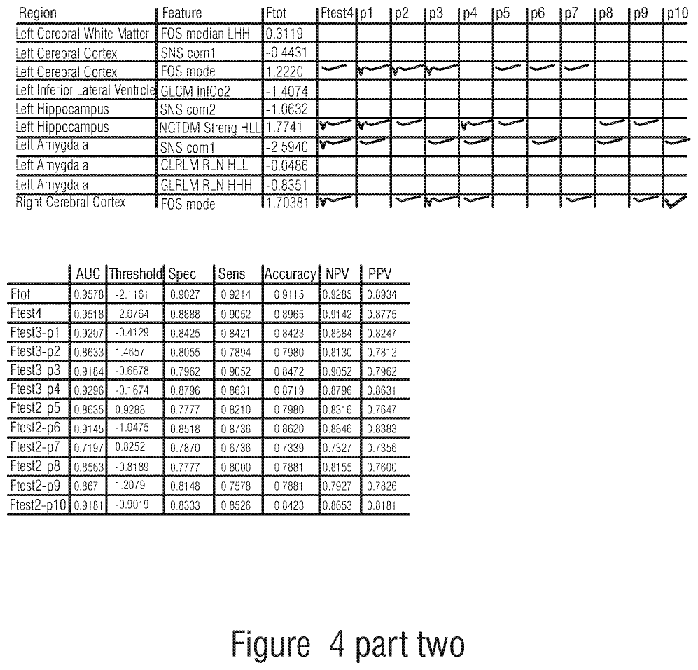

In the first study, the indicated combinations of features from the rows listed in Table 1 were selected, and tested using an ROC analysis. The complete ROC data is shown in FIG. 4, but summarised below using the AUC (area under curve) obtained from the ROC analysis of each feature set.

TABLE 7

| | Rows of | ROC- |
|---|---|---|
| Combination | Table 1 | AUC |
| Ftot | All | 0.9578 |
| Ftest4 | c, f, g, j | 0.9518 |
| P1 | c, f, g | 0.9207 |
| P2 | c, f, j | 0.8633 |
| P3 | c, g, j | 0.9184 |
| P4 | f, g, j | 0.9296 |
| P5 | c, f | 0.8635 |
| P6 | c, g | 0.9145 |
| P7 | c, j | 0.7197 |
| P8 | f, g | 0.8563 |
| P9 | f, j | 0.867 |
| P10 | g, j | 0.9181 |

The performance achieved using only four of the heavily weighted feature-region tuples (Ftest4) is closely comparable with that achieved by using the entire feature set listed in Table 1. Where only three features are used, effective performance is still achieved and the best performance is given by permutation 4 (P4), which involves he Left Hippocampus, Left Amygdala and Right Cerebral Cortex—see rows f, g and j of Table 1.

Where only two of the feature-region tuples were used, the best performance was provided by the permutation labelled P6, which involves Left Cerebral Cortex and Left Amygdala—see rows c and g of Table 1. The worst performance was given by the combination of Left and Right Cerebral Cortex alone, labelled P7. Even this however provided a reasonable degree of class separation, so a measurable predictive effect is present even in this less preferred embodiment.

It can thus be seen that whilst there are some particularly advantageous combinations, selection of any two of the four heavily weighted feature-region tuples listed above provides effective prediction or diagnosis of cognitive impairment. The full table with complete ROC data is set out in FIG. 4.

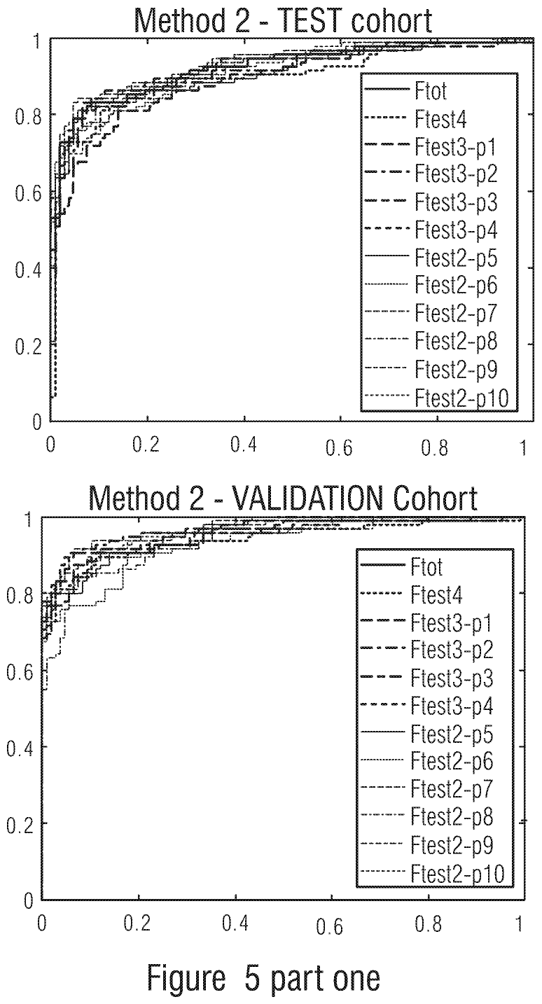

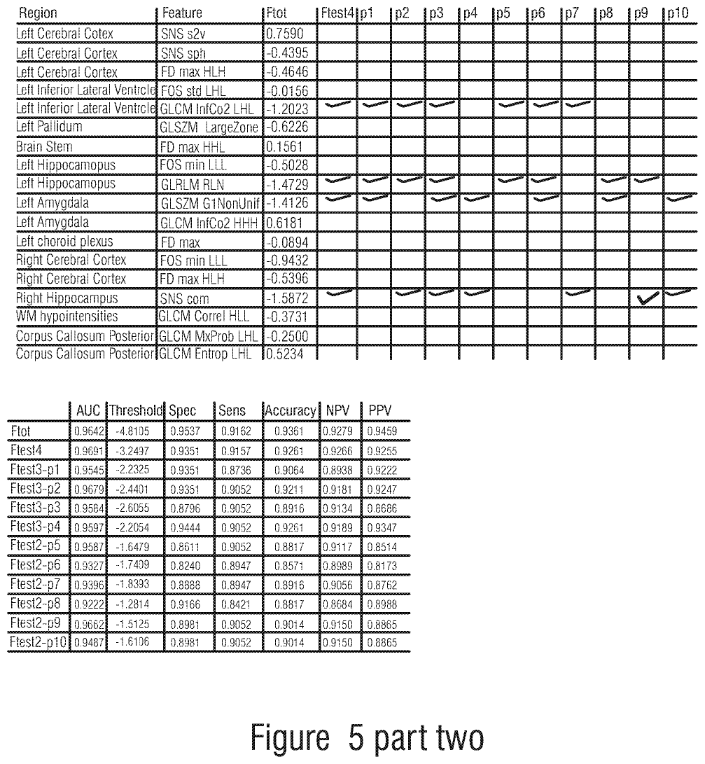

In the second study, combinations of features from the rows listed in Table 2 were selected and tested using an ROC analysis. The complete ROC data is shown in FIG. 5, but is summarised below using the AUC (area under curve) obtained from the ROC analysis of each feature set.

TABLE 8

| | Rows of | ROC- |
|---|---|---|
| Combination | Table 2 | AUC |
| Ftot | All | 0.9642 |
| Ftest4 | E, I, J, O | 0.9691 |
| P1 | E, I, J | 0.9545 |
| P2 | E, I, O | 0.9679 |
| P3 | E, J, O | 0.9584 |
| P4 | I, J, O | 0.9597 |
| P5 | E, I, | 0.9587 |
| P6 | E, J | 0.9327 |
| P7 | E, O | 0.9396 |
| P8 | I, J | 0.9222 |
| P9 | I, O | 0.9662 |
| P10 | J, O | 0.9487 |

As with the reduced feature sets explained with reference to Table 7, Table 8 also shows that the performance achieved using only four of the heavily weighted feature-region tuples from Table 2 (Ftest4) is closely comparable with that achieved by using the entire feature set of Table 2.

Where only three features are used, effective performance is still achieved and the best performance is given by permutation 4 (P4), which involves the Left Hippocampus, Left Amygdala and Right Hippocampus—see rows I, J and O of Table 2.

Where only two of the feature-region tuples were used, the best performance was provided by the permutation labelled P9, which involves Left Hippocampus and Right Hippocampus—see rows I and O of Table 2. The performance of all of the reduced sets using only two feature-region tuples of Table 2 provided AUC performance of well above 0.9

It can thus be seen that whilst there are some particularly advantageous combinations, selection of any two of the four heavily weighted feature-region tuples listed above provides very effective prediction or diagnosis of cognitive impairment. The full table with complete ROC data is set out in FIG. 5.

### The Use of Sub-Regions of the Cortex

In addition, or as an alternative to, the use of the image regions outlined above embodiments of the present disclosure comprise methods which identify regions of interest ROIs comprising sub-regions of the cortex (which may be referred to as sub cortical regions). Such methods of predicting or diagnosing cognitive impairment may find particular application in providing a visual guide to the stage of cognitive impairment and/or the progress of disease states such as Alzheimer's disease (AD).

The apparatus which performs these methods may be identical to that outlined above, other than in that it may comprise a display means for providing an overlay of the results of the methods disclosed herein on an image of a subject's brain and/or on a standard brain such as an anatomical atlas.

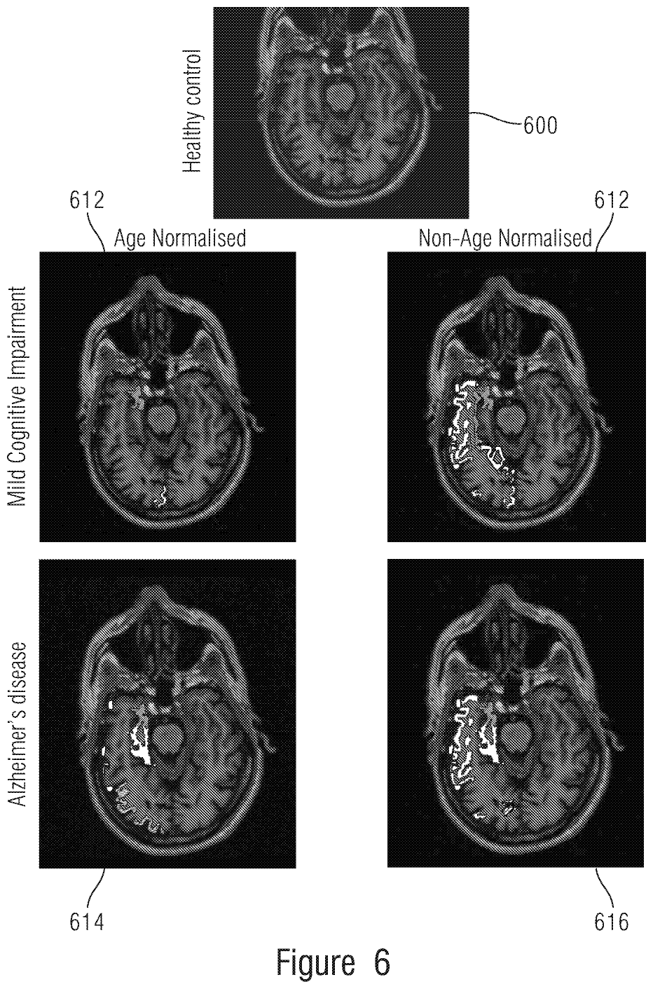

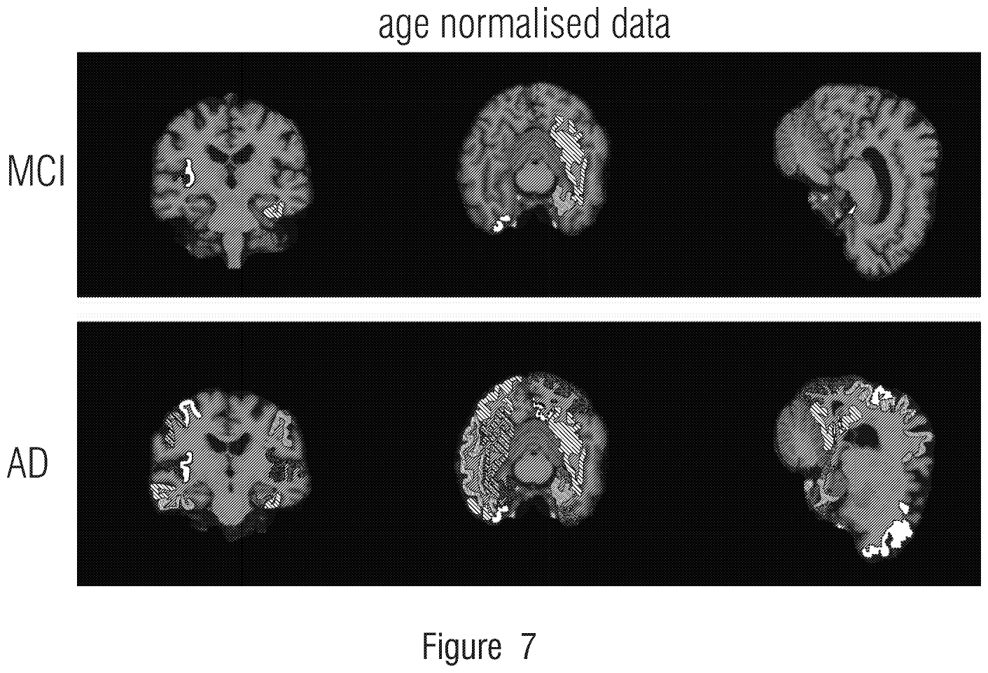

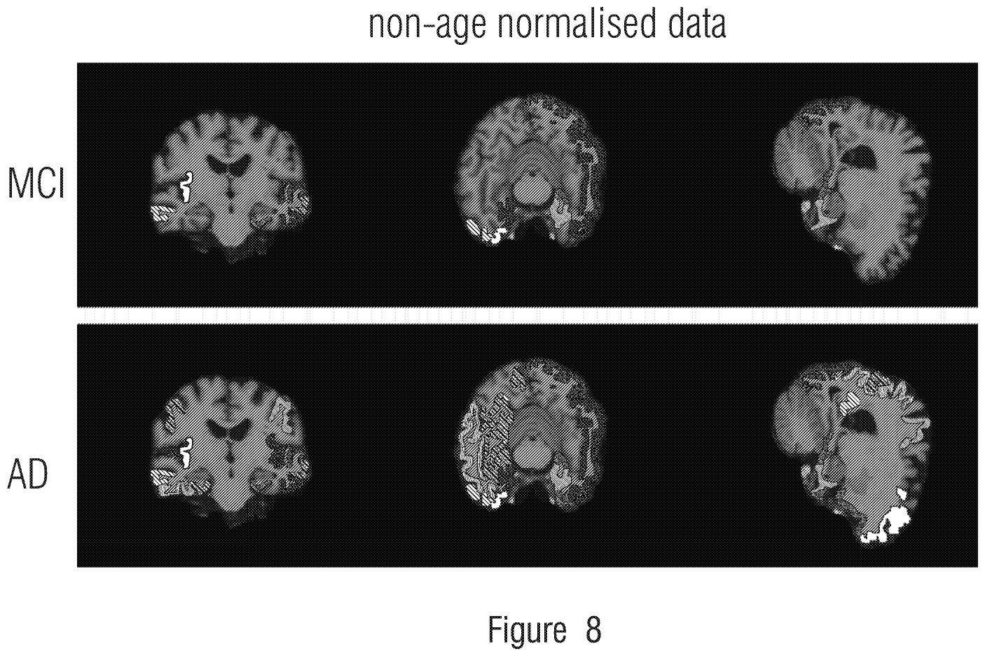

FIG. 6, FIG. 7 and FIG. 8 illustrate the type of visual output which may be obtained from such methods FIG. 7 relates to the data obtained from mild cognitive impairment (MCI) and alzheimer's patients (AD) in age normalised image data. FIG. 8 relates to the data obtained from mild cognitive impairment (MCI) and alzheimer's patients (AD) in non-age normalised image data. Such visual indications may be determined by calculating the feature/region tuples defined in any one of Tables 9 to 13 below, and displaying the results as an overlay on an anatomical/structural image of the human brain to assist a clinician in diagnosing/stratifying patients.

The controller may also be configured to identify, in image data having appropriate contrast and resolution, certain sub-regions of the cortex. The sub-regions of the cortex may comprise:
Banks of the Superior Temporal Sulcus
Caudal Middlefrontal
Entorhinal
Frontal pole
Fusiform
Inferior parietal
Inferior temporal
Insula
Isthmus cingulate
Lateral Occipital
Lateral Orbitofrontal
lingual
Middle temporal
Parahippocampal
Pars Orbitalis
Pars Triangularis
Pericalcarine
Precentral
Precuneus
Rostral anterior cingulate
Rostral middlefrontal
Superior parietal
Superior temporal
Supramarginal
Temporal pole
Transverse Temporal

In the embodiments in which these sub regions of the cortex are used, the controller is able to identify both the left and right brain locations of these regions. The image metrics employed in each of these regions may comprise any one or more of those outlined above. The image data may be age normalised as described above.

In particular embodiments, the controller is configured to identify: the left entorhinal, left fusiform and the right temporal pole, and the transverse temporal pole. In each of these regions the controller then determines one or more of the following image metrics:
a metric of image texture;
a metric of image intensity; and
a morphological metric of the image region.

Different implementations can be used—and some of the metrics which have been found to be of particularly strong predictive power are outlined in Table 9, Table 10, Table 11, and Table 12, below. It can be seen from these different tables of data that, in addition to the consistent usefulness of these four regions, the insula was also found to play a significant part in some approaches.

Table 9, below, sets out one set of tuples of sub-cortical regions, and the image metrics for each region. These have been applied to age normalised data and found to have a particularly strong predictive effect. The weightings listed can be used to combine the image metrics from the identified regions, for example in a linear sum. Other predetermined methods may also be used. The resultant indicator may be used in the prediction and/or diagnosis of cognitive impairment as described above.

In addition to the quantitative predictive/diagnostic effectiveness of these methods, it has also been found that overlays of the relevant image metrics on images of the human brain provide a useful marker of the extent of cognitive impairment. Examples of such overlays are illustrated in FIG. 6. The display of visual indicators of the metrics of sub-regions of the cortex as defined herein may therefore provide a useful diagnostic aide, to assist clinicians in making an assessment of cognitive impairment in a subject.

TABLE 9

| Region | Feature | Weight |
|---|---|---|
| L Entorhinal | shape and size surface to | 3.295269 |
| | spherical disproportion | |
| L Entorhinal | GLSZM small zone high grey level | 1.987232 |
| | emphasis with HHL filter | |
| L Entorhinal | GLSZM small zone high grey level | 1.365442 |
| | emphasis with LHL filter | |
| L Entorhinal | GLCM difference variance | 1.319203 |
| | with LHH filter | |
| L Entorhinal | GLRLM short run emphasis | 0.379947 |
| L Entorhinal | GLSZM small zone high grey level | 0.053123 |
| | emphasis with HLL filter | |
| L Fusiform | GLRLM short run length low grey | 3.272863 |
| | level emphasis with LHL filter | |
| L Fusiform | First Order Statistics | 1.28828 |
| | mode with LHL filter | |
| L Fusiform | first order statistics | 5.42E−06 |
| | minimum with LHL filter | |
| R Insula | GLRLM short run emphasis | 2.922947 |
| | with LLL filter | |
| R Temporalpole | First Order Statistics | −1.70099 |
| | median with LLH filter | |
| R Temporalpole | First Order Statistics | −0.58523 |
| | mean with LLH filter | |
| R Transverse | Mean Absolute Deviation | −3.11412 |
| Temporal | with LHH filter | |
| R Transverse | mean fractal dimension | −2.88058 |
| Temporal | with HLL filter | |
| R Transverse | First Order Statistics | 1.712749 |
| Temporal | mode with HHH filter | |
| R Transverse | Mean Absolute Deviation | −0.18015 |
| Temporal | with HHH filter | |
| R Transverse | First Order Statistics | 6.43E−06 |
| Temporal | minimum with HHH filter | |

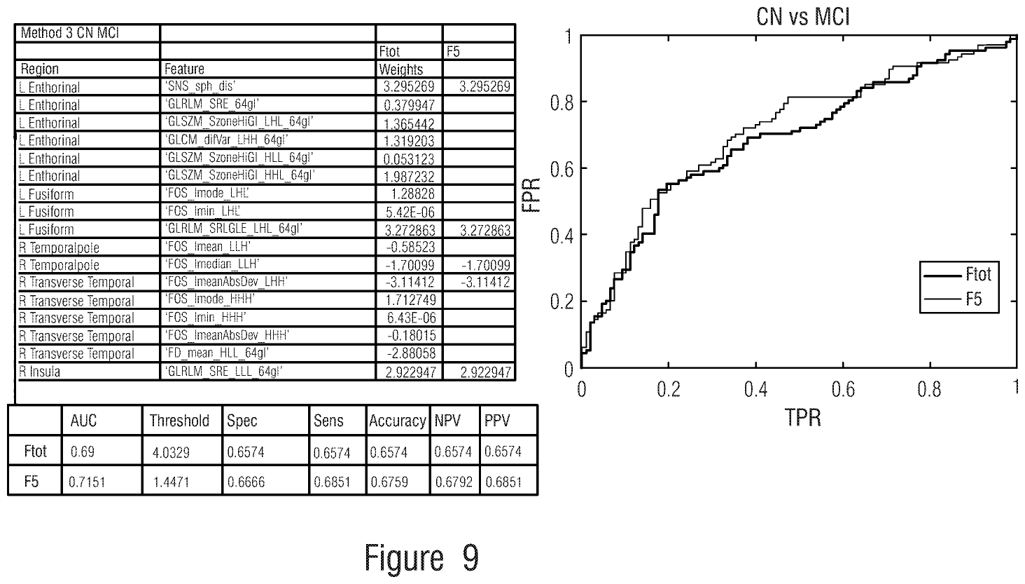

An extract of the ROC analysis of using this approach to distinguish between age normalised images of control subjects and subjects having mild cognitive impairment (MCI) is illustrated in FIG. 9.

It is strongly indicated by the data set out above in Table 7 and Table 8, that when combining image metrics from different regions in this way, the predictive power of the method may remain even when using reduced feature sets. This hypothesis was tested by making comparative assessments of the predictive power of the method defined in Table 9, above. In this comparative test, the predictive power of the full feature set listed in Table 9 was compared against the power of a reduced feature set in which only 5 tuples were selected. The tuples chosen for the comparative test were:
1. a morphological feature of the left entorhinal (e.g. spherical distortion)
2. a texture feature of the left fusiform (e.g. GLRLM short run low grey level emphasis)
3. an image intensity of the right temporal pole (median intensity)
4. an image intensity of the right transverse temporal cortex—(e.g. mean absolute deviation, which may comprise the mean distance of all intensity values from the Mean Value of the image array

$$Mean Abs Dev = \frac{1}{{N}_{P}} {\sum }_{i = 1}^{N p} \vert X ( i ) - \overline{X} \vert ; and$$

5. a texture feature of the right insula (e.g. GLRLM short run emphasis)

It can be seen from FIG. 9 that the performance of the reduced feature set (labelled F5 in FIG. 9) which uses just five (5) of the 17 tuples listed in Table 9 provides a predictive/diagnostic effect nearly equivalent to that provided by the full feature set. This is consistent with the observations set out above—namely that a reduced feature set using the more strongly weighted feature/region tuples provides effective performance.

It can be seen from an inspection of Table 9, above, that whilst certain texture, intensity, and morphology features have been selected in each of these regions other such features also provide significant contributions to the predictive power of the approach. It is therefore believed that the combination of the particular image regions, and texture/morphology/intensity based image metrics derived from those regions provides the predictive/diagnostic effect of the present disclosure. The precise detail of the metrics themselves is a lesser consideration than the underlying anatomical/physiological effect which they reveal. Other image metrics of texture, morphology and so forth may be used.

A further predictor was derived using control patients and patients with Alzheimer's Disease. The data used to establish this model was age normalised in the manner described above. The feature/region tuples used in this predictor are set out below in Table 10

TABLE 10

| Regions | long name | Weights |
|---|---|---|
| L Banks of the | GLSZM small zone low grey | 0.170415 |
| Superior | level with LLL filter | |
| Temporal Sulcus | | |
| L Caudal | grey level size zone matrix | 0.144365 |
| Middlefrontal | small zone emphasis with LHL filter | |
| L Enthorinal | shape and size surface to | 3.049285 |
| | volume ratio | |
| L Enthorinal | GLSZM zone percentage with | 0.956581 |
| | LLL filter | |
| L Enthorinal | NGTDM busyness with LLH filter | −0.77649 |
| L Enthorinal | GLSZM small zone high grey level | 0.229988 |
| | emphasis with HLL filter | |
| L Enthorinal | GLCM Correlation with | −0.01224 |
| | HHH filter | |
| L Enthorinal | shape and size surface | −0.00135 |
| | to sphericity | |
| L Frontalpole | First Order Statistics mean | 0.153934 |
| | with HHL filter | |
| L Fusiform | GLSZM grey level variance | 0.46578 |
| L Fusiform | mean fractal dimension | −0.41496 |
| | with HHL filter | |
| L Fusiform | NGTDM complexity with LLL filter | 0.178951 |
| L Fusiform | GLCM cluster prominence | 0.022571 |
| | with LLH filter | |
| L Inferiorparietal | GLRLM run length nonuniformity | −2.95444 |
| L Inferiorparietal | GLCM information based measure of | −0.0713 |
| | correlation 1 with HLL filter | |
| L Insula | First Order Statistics mode with | −2.74864 |
| | HLL filter | |
| L Insula | variance of the fractal dimension | −0.63036 |
| | with a HHL filter | |
| L Insula | first order statistics minimum | −0.00016 |
| | with HLL filter | |
| L Isthmus | maximum fractal dimension | −0.48581 |
| cingulate | with HHH filter | |
| L Isthmus | Grey level co-occurrence matrix | −0.30605 |
| cingulate | joint entropy with a HLH filter | |
| L Isthmus | First Order Statistics mode | −0.03194 |
| cingulate | with LLH filter | |
| L Isthmus | first order statistics minimum | −1.24E−05 |
| cingulate | with LLH filter | |
| L Lateral | fractal dimension standard | −2.1886 |
| Occipital | deviation with LHL filter | |
| L lingual | grey level size zone matrix | 0.314208 |
| | size zone non uniformity | |
| | with LLL filter | |
| L lingual | GLCM cluster shade with HLL filter | 0.089456 |
| L lingual | Grey level co-occurrence matrix | −0.01972 |
| | maximum probability with LLH filter | |
| L Parahippocampal | shape and size, compactness 1 | −0.94001 |
| L Parahippocampal | NGTDM busyness with LHL filter | −0.61542 |
| L Parahippocampal | minimum fractal dimension | 0.400015 |
| | with LLL filter | |
| L Parahippocampal | NGTDM strength with LLL filter | 0.091937 |
| L ParsOrbitalis | First Order Statistics mean | −0.49261 |
| | with HLL filter | |
| L Parstriangularis | First Order Statistics mean | 1.283595 |
| | with HLH filter | |
| L Precentral | GLCM difference entropy | 0.617321 |
| L Precentral | GLSZM small zone high grey level | 0.053608 |
| | emphasis with LLL filter | |
| L Precuneus | GLCM information based measure | −0.6645 |
| | of correlation with LHL filter | |
| L Rostral | GLSZM zone size variance | 0.964688 |
| middlefrontal | | |
| L Rostral | NGTDM strength with HHH filter | 0.598154 |
| middlefrontal | | |
| L Superiorparietal | GLSZM grey level variance | 0.08388 |
| | with HLL filter | |
| L Superiortemporal | GLSZM zone low grey level | 1.521496 |
| | emphasis with LLH filter | |
| L Superiortemporal | First Order Statistics maximum | −1.35223 |
| | with LLL filter | |
| L Superiortemporal | Grey level co-occurrence matrix | 1.115139 |
| | sum average with HHH filter | |
| L Superiortemporal | First Order Statistics skewness | 0.494638 |
| | with HLL filter | |
| L Supramarginal | GLSZM zone high grey level | −2.20863 |
| | emphasis with HLL filter | |
| L Supramarginal | First Order Statistics median | 0.211957 |
| | with HLH filter | |
| L Temporalpole | GLCM cluster prominence with | 0.264933 |
| | LHL filter | |
| L Temporalpole | GLCM sum entropy with LLL filter | −0.17607 |
| R Banks of the | First Order Statistics median | −1.48796 |
| Superior | with LLL filter | |
| Temporal Sulcus | | |
| R Banks of the | Grey level co-occurrence matrix | −1.15302 |
| Superior | joint entropy with a LLH filter | |
| Temporal Sulcus | | |
| R Banks of the | Grey level co-occurrence matrix | −0.70663 |
| Superior | joint entropy with a LHL filter | |
| Temporal Sulcus | | |
| R Banks of the | maximum fractal dimension | −0.61167 |
| Superior | with HHL filter | |
| Temporal Sulcus | | |
| R Banks of the | GLCM difference entropy | 0.199848 |
| Superior | with LLL filter | |
| Temporal Sulcus | | |
| R Banks of the | GLSZM zone percentage with | 0.17245 |
| Superior | LLL filter | |
| Temporal Sulcus | | |
| R Banks of the | GLCM information based measure of | 0.032353 |
| Superior | correlation with HLL filter | |
| Temporal Sulcus | | |
| R Banks of the | First Order Statistics maximum | −0.02894 |
| Superior | with LLL filter | |
| Temporal Sulcus | | |
| R Caudal | GLSZM zone size variance | 1.102021 |
| Middlefrontal | with LLL filter | |
| R Enthorinal | shape and size, | −1.10584 |
| | compactness 2 | |
| R Enthorinal | GLSZM zone percentage with | 1.012529 |
| | LLL filter | |
| R Enthorinal | minimum fractal dimension | 0.64708 |
| | with LHL filter | |
| R Enthorinal | GLRLM long run emphasis | −0.51931 |
| R Enthorinal | grey level size zone matrix small | 0.470111 |
| | zone emphasis with LLH filter | |
| R Enthorinal | GLCM information based measure | 0.312182 |
| | of correlation with LLH filter | |
| R Enthorinal | shape and size surface to | 0.105788 |
| | volume ratio | |
| R Fusiform | maximum fractal dimension | −1.8349 |
| | with LHL filter | |
| R Inferiorparietal | GLSZM large zone high grey level | −1.28437 |
| | emphasis with LLL filter | |
| R Inferiorparietal | GLCM information based measure of | −1.20131 |
| | correlation 1 with HHL filter | |
| R Inferiortemporal | GLRLM high grey level long run | −0.52235 |
| | emphasis with HHL filter | |
| R Insula | First Order Statistics mode | 2.524577 |
| | with LLL filter | |
| R Insula | First Order Statistics mean | 0.507135 |
| | with LHH filter | |
| R Insula | first order statistics minimum | 0.000107 |
| | with HLL filter | |
| R LateralOccipital | minimum fractal dimension | 1.137846 |
| | with LLL filter | |
| R Middletemporal | grey level size zone matrix size zone | −2.25961 |
| | non uniformity with HHL filter | |
| R Pars | mean fractal dimension | 3.920725 |
| Triangularis | with LLL filter | |
| R Pars | lacunarity of fractal dimension | −0.4736 |
| Triangularis | with LLL filter | |
| R Precentral | First Order Statistics median | −0.56497 |
| | with LHL filter | |
| R Precuneus | mean fractal dimension with | 1.061564 |
| | LLL filter | |
| R Supramarginal | GLSZM small zone high grey level | 0.113365 |
| | emphasis with LHL filter | |
| R Temporalpole | minimum fractal dimension | 1.981675 |
| | with LHH filter | |
| R Transverse | First order statistics mean absolute | −0.83475 |
| Temporal | deviance with HLL filter | |
| R Transverse | GLRLM short run high grey level | −0.48063 |
| Temporal | emphasis with HHH filter | |
| R Transverse | First Order Statistics maximum | −0.47761 |
| Temporal | with LHL filter | |

Significantly, it can be seen that a morphological feature of the entorhinal cortex is again very heavily weighted in this data. In this instance the parameter is the surface to volume ratio, but it will be appreciated in the context of the present disclosure that such parameters are not unrelated to the sphericity of a region, or indeed the spherical disproportion. Embodiments of the present disclosure may thus comprise methods of predicting or diagnosing cognitive impairment based on morphological features of the entorhinal cortex.

Other features which are heavily weighted in Table 10 are also heavily weighted in Table 9. For example the insula, the fusiform, and the temporal pole amongst other regions are heavily weighted in both Table 9 and Table 10.

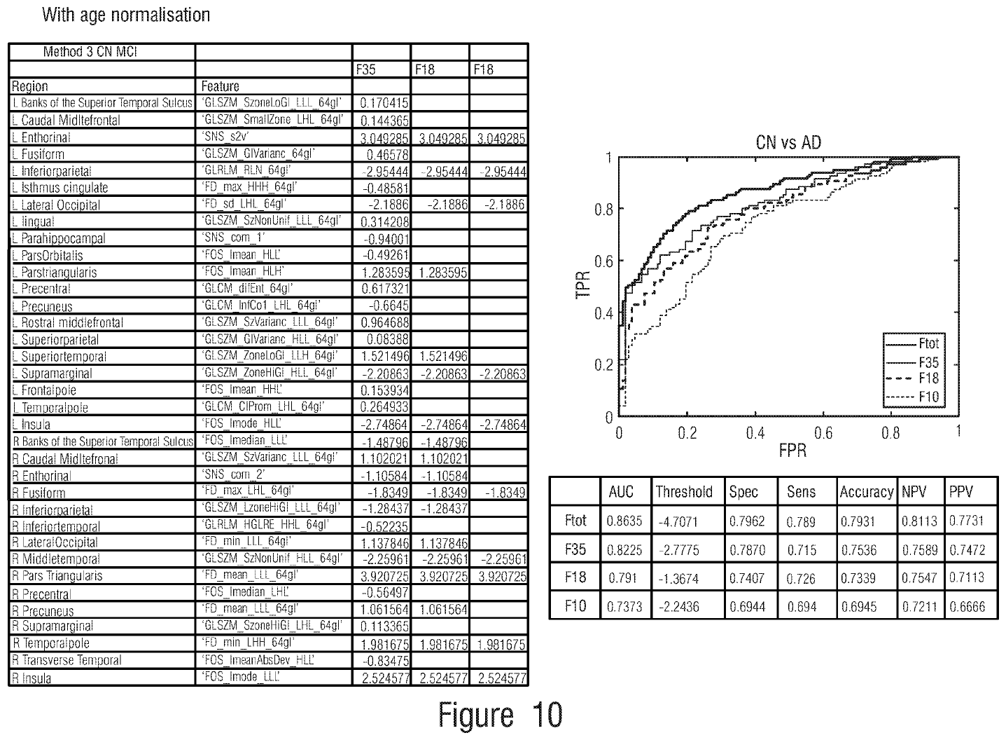

To test the hypothesis that the most strongly weighted feature/region tuples can provide an effective predictor without use of the full feature set, a set of comparative tests were performed. In these comparative tests, the full 80 tuple feature set defined in Table 10 was compared against the reduced feature sets shown in FIG. 10.

In FIG. 10, Fₜₒₜ indicates the full 80 tuple feature set. The following feature sets are also defined:
F35: 35 features correspondent to the 35 highest weighted features per region
F18: the 18 highest weighted among the 35
F10: the 10 highest weighted among the 18

An ROC analysis was performed to compare the predictive/diagnostic power of the full feature set against the reduced sets. It can be seen that even the 10 tuple feature set, F10, provides an effective predictor with an AUC of 0.73. Again, the entorhinal, temporal pole, and insula provide a significant contribution to the effectiveness of this predictor.

Table 11, below shows the feature/region tuples developed for a non-age normalised data set comprising control patients and patients with mild cognitive impairment (MCI)

TABLE 11

| Regions | long name | Weights |
|---|---|---|
| L Caudal | NGTDM complexity with | −1.31758 |
| Middltefrontal | LHL filter | |
| L Caudal | GLCM difference variance | 0.116458 |
| Middltefrontal | | |
| L Enthorinal | GLCM Correlation with | 1.540307 |
| | HLL filter | |
| L Enthorinal | maximum fractal dimension | −0.95594 |
| | with LHL filter | |
| L Enthorinal | GLSZM small zone emphasis | 0.950351 |
| L Enthorinal | minimum fractal dimension | 0.695691 |
| | with HHL filter | |
| L Enthorinal | First Order Statistics mean | −0.60892 |
| | with LHL filter | |
| L Enthorinal | minimum fractal dimension | 0.176888 |
| L Enthorinal | GLCM inverse variance | −0.1582 |
| L Enthorinal | maximum fractal dimension | −0.13014 |
| | with HHH filter | |
| L Inferiortemporal | First Order Statistics RMS | 0.242942 |
| | with HLL filter | |
| L Isthmus | First Order Statistics mean | −0.66677 |
| cingulate | with HLL filter | |
| L LateralOccipital | NGTDM complexity with HHL filter | 2.161983 |
| L Middletemporal | GLCM Contrast | 0.343334 |
| L Middletemporal | GLSZM large zone low grey level | −0.24811 |
| | emphasis with HHH filter | |
| L Parahippocampal | Grey level co-occurrence matrix | 0.322795 |
| | sum variance with LHH filter | |
| L Parahippocampal | Grey level co-occurence matrix | 5.30E−10 |
| | cluster tendency with LHH filter | |
| L ParsOrbitalis | mean fractal dimension | 1.38805 |
| L Superiortemporal | maximum fractal dimension | −1.10252 |
| | with HHL filter | |
| L Superiortemporal | mean fractal dimension | 0.150033 |
| L Superiortemporal | NGTDM complexity | 0.022989 |
| R Banks of the | mean fractal dimension with | −2.40888 |
| Superior | LHL filter | |
| Temporal Sulcus | | |
| R Banks of the | Grey level co-occurrence matrix | 0.742583 |
| Superior | sum variance with HHL filter | |
| Temporal Sulcus | | |
| R Banks of the | First Order Statistics | −0.60401 |
| Superior | median with LLH filter | |
| Temporal Sulcus | | |
| R Banks of the | Grey level co-occurence matrix | 2.78E−10 |
| Superior | cluster tendency with HHL filter | |
| Temporal Sulcus | | |
| R Enthorinal | shape and size, compactness 2 | −1.15717 |
| R Enthorinal | Grey level co-occurrence | 0.318659 |
| | matrix sum variance | |
| R Enthorinal | First Order Statistics mean | −0.19651 |
| | with LLH filter | |
| R Enthorinal | Grey level co-occurence | 0.000878 |
| | matrix cluster tendency | |
| R Inferiorparietal | minimum fractal dimension | 0.90721 |
| | with HHL filter | |
| R Insula | minimum fractal dimension | 0.584533 |
| | with HLL filter | |
| R Insula | First Order Statistics mean | 0.129373 |
| | with HHL filter | |
| R Middletemporal | GLSZM small zone high grey level | 0.745496 |
| | emphasis with HHL filter | |
| R Middletemporal | minimum fractal dimension | 0.165184 |
| | with HLL filter | |
| R Pars Orbitalis | NGTDM strength with LLL filter | 0.538842 |
| R Pars Orbitalis | NGTDM busyness | −0.35922 |
| R Rostral | First Order Statistics | 1.051034 |
| Middlefrontal | median with HHL filter | |
| R Rostral | GLSZM small zone low grey | 0.323497 |
| Middlefrontal | level with LLL filter | |
| R Temporalpole | GLSZM small zone low grey | 0.709318 |
| | level with LLL filter | |
| R Temporalpole | GLSZM large zone high grey | −0.23521 |
| | level emphasis with HHH filter | |
| R Temporalpole | GLSZM large zone emphasis | −0.20974 |
| | with HLH filter | |

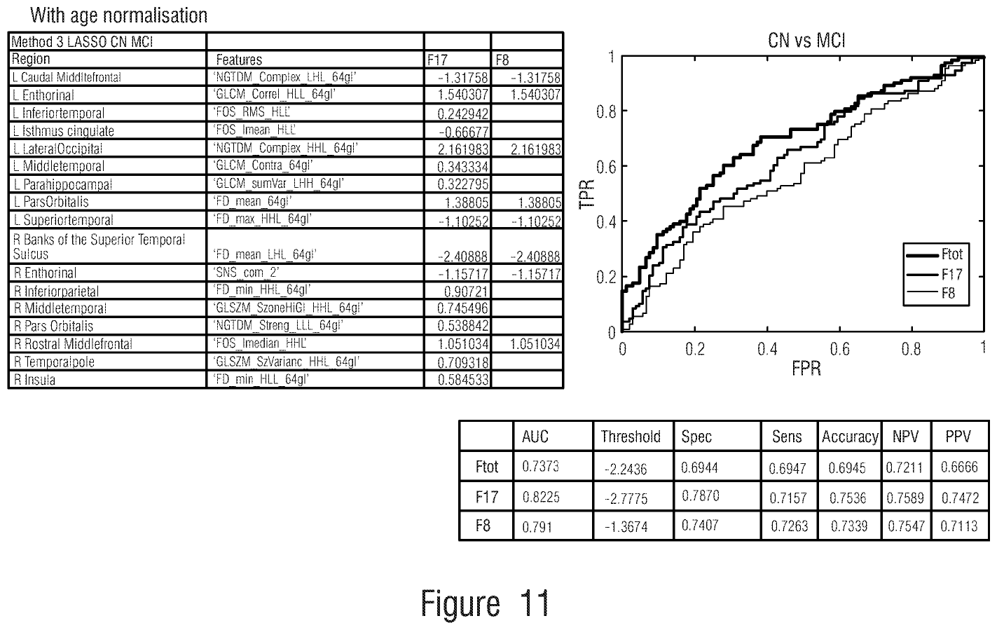

It can be seen that this analysis gives rise to 41 feature/region tuples in 17 regions of the brain. A comparative test was performed in which the performance of a predictor based on 17 tuples (the highest weighted feature for each region) was tested against the full feature set and a further reduced feature set was also used in which only the 8 highest weighted tuples from amongst that 17 were used. These are labelled Fₜₒₜ, F17, and F8 respectively in FIG. 11. It can be seen from the ROC analysis presented in FIG. 11 that both reduced feature sets provide effective predictive/diagnostic power.

It can also be seen from a comparison of the performance of the predictive models defined in Tables 9 and 11 that although age normalisation may have some advantages it is by no means essential. An effective prediction can be made without it. In addition the morphology of the entorhinal cortex again plays a significant role—in this instance the compactness.

Table 12 below shows the feature/region tuples for a predictor developed from a data set comprising control patients and patients with Alzheimer's Disease. The image data was not age normalised.

| Regions | Features | Weights |
|---|---|---|
| L Banks of the | GLCM information based | 0.888641 |
| Superior | measure of correlation | |
| Temporal Sulcus | with LHL filter | |
| L Banks of the | mean fractal dimension | 0.870555 |
| Superior | with HHL filter | |
| Temporal Sulcus | | |
| L Banks of the | First order statistics | 0.740675 |
| Superior | mean absolute deviance | |
| Temporal Sulcus | with HLL filter | |
| L Enthorinal | neighbourhood grey tone | 2.559715 |
| | difference matrix (NGTDM) | |
| | contrast, with LLL filter | |
| L Enthorinal | Grey level run length | 1.624852 |
| | matrix long run length | |
| | low grey level emphasis | |
| L Enthorinal | GLCM difference variance | 1.557292 |
| L Enthorinal | GLCM information based | 1.076048 |
| | measure of correlation | |
| | with HLH filter | |
| L Enthorinal | GLRLM short run high | 0.916666 |
| | grey level emphasis | |
| | with HHH filter | |
| L Enthorinal | GLSZM small zone high | 0.727289 |
| | grey level emphasis | |
| L Enthorinal | GLSZM grey level variance | −0.34558 |
| L Enthorinal | GLCM information based | 0.127109 |
| | measure of correlation | |
| L Enthorinal | GLCM information based | 0.053807 |
| | measure of correlation | |
| | with HLL filter | |
| L Enthorinal | GLSZM small zone high | 0.005602 |
| | grey level emphasis | |
| | with HHH filter | |
| L Frontalpole | GLSZM small zone high | 0.536633 |
| | grey level emphasis | |
| | with HLL filter | |
| L Inferioritemporal | First Order Statistics | 3.526849 |
| | RMS with HLL filter | |
| L Inferioritemporal | First Order Statistics | 0.178556 |
| | maximum with HLL filter | |
| L Inferioritemporal | First Order Statistics | −0.0899 |
| | median with HLL filter | |
| L Inferiorparietal | GLRLM run length | −0.73427 |
| | nonuniformity | |
| | with LHH filter | |
| L Inferiorparietal | First Order Statistics | −0.53161 |
| | Energy with HHH filter | |
| L Inferiorparietal | GLSZM small zone high | 0.48923 |
| | grey level emphasis | |
| | with LHH filter | |
| L Insula | GLRLM short run high | 0.995209 |
| | grey level emphasis | |
| | with HHH filter | |
| L Isthmus | First Order Statistics | −1.65017 |
| cingulate | median with LLH filter | |
| L Lateral | First Order Statistics | 0.225136 |
| Orbitofrontal | maximum with LLH filter | |
| L Middletemporal | maximum fractal dimension | −2.19676 |
| | with HHL filter | |
| L Middletemporal | GLRLM run percentage | 1.499056 |
| | with HLL filter | |
| L Parahippocampal | First Order Statistics | 0.620384 |
| | skewness with LHL filter | |
| L Parahippocampal | GLCM information based | 0.595259 |
| | measure of correlation | |
| | with LLL filter | |
| L Parahippocampal | First Order Statistics | 0.348604 |
| | maximum with HLL filter | |
| L Parahippocampal | GLCM Correlation | 0.134036 |
| | with LHH filter | |
| L Parahippocampal | GLSZM grey level variance | −0.01686 |
| | with HLL filter | |
| L ParsOrbitalis | mean fractal dimension | 0.588439 |
| | with LHL filter | |
| L Precentral | maximum fractal dimension | 1.278405 |
| | with HHH filter | |
| L Precentral | GLCM grey level variance | 0.117173 |
| L Precuneus | maximum fractal dimension | −1.56941 |
| | with LHL filter | |
| L Rostral | grey level size zone | 1.777615 |
| middlefrontal | matrix small zone emphasis | |
| L Rostral | GLRLM long run high grey | −0.07205 |
| middlefrontal | level emphasis with HHL filter | |
| L Rostral | minimum fractal dimension | 0.064977 |
| middlefrontal | | |
| L | First Order Statistics | 0.47461 |
| Rostralanteriorcingulate | maximum with HHL filter | |
| L Superiorparietal | grey level co-occurrence | 2.218497 |
| | matrix autocorrelation | |
| | with HHL filter | |
| L Supramarginal | GLRLM short run high | 0.040094 |
| | grey level emphasis | |
| | with LHH filter | |
| L Temporalpole | First Order Statistics | 0.867909 |
| | RMS with LLH filter | |
| L Temporalpole | Grey level co-occurrence | −0.61069 |
| | matrix sum average | |
| | with HLL filter | |
| R Banks of the | maximum fractal dimension | −0.48825 |
| Superior | with LLL filter | |
| Temporal Sulcus | | |
| R Banks of the | GLSZM small zone low | 0.242826 |
| Superior | grey level emphasis | |
| Temporal Sulcus | | |
| R Enthorinal | GLCM information based | 1.475704 |
| | measure of correlation | |
| | with LLH filter | |
| R Enthorinal | grey level size zone | 1.042112 |
| | matrix small zone | |
| | emphasis with LLL filter | |
| R Enthorinal | maximum fractal dimension | −0.81562 |
| | with LHL filter | |
| R Enthorinal | GLCM difference variance | 0.124307 |
| R Enthorinal | GLCM Correlation | 0.109949 |
| | with HHH filter | |
| R Enthorinal | Grey level co-occurrence | −0.04415 |
| | matrix inverse different | |
| | moment normalised | |
| R Frontalpole | First order statistics | 1.197703 |
| | entropy with HHL filter | |
| R Frontalpole | GLSZM small zone low | 0.939372 |
| | grey level with HLH filter | |
| R Frontalpole | GLSZM zone low grey level | 0.727302 |
| | emphasis with HLH filter | |
| R Frontalpole | GLRLM short run length | 0.109486 |
| | low grey level emphasis | |
| | with LHH filter | |
| R Fusiform | NGTDM busyness with | −1.23079 |
| | HLL filter | |
| R Fusiform | First Order Statistics | 0.278442 |
| | median with HHH filter | |
| R Fusiform | GLSZM grey level variance | −0.03732 |
| R Inferiorparietal | GLSZM small zone high | 0.668561 |
| | grey level emphasis | |
| | with HLH filter | |
| R Inferiortemporal | GLSZM small zone emphasis | 0.423731 |
| | with HHH filter | |
| R Isthmus | First Order Statistics | −0.85146 |
| cingulate | mode with HLL filter | |
| R Isthmus | GLCM Autocorrelation | −0.79826 |
| cingulate | with HHH filter | |
| R Isthmus | first order statistics | −4.12E−06 |
| cingulate | minimum with HLL filter | |
| R Lateral | GLSZM zone size variance | −1.56146 |
| Orbitofrontal | | |
| R Middletemporal | GLSZM small zone high | 0.215538 |
| | grey level emphasis | |
| | with HHL filter | |
| R Parahippocampal | maximum fractal dimension | 0.741955 |
| | with HHL filter | |
| R Parahippocampal | GLCM information based | 0.65008 |
| | measure of correlation | |
| | with HLH filter | |
| R Parahippocampal | Grey level co-occurrence | −0.6041 |
| | matrix inverse different | |
| | moment normalised | |
| | with HLH filter | |
| R Parahippocampal | GLCM difference variance | −0.16653 |
| | with HLL filter | |
| R Parahippocampal | maximum fractal dimension | −0.07323 |
| R Pars Orbitalis | Grey level co-occurrence | 0.187851 |
| | matrix sum variance | |
| | with LHL filter | |
| R Pars Orbitalis | GLCM cluster prominence | 0.167818 |
| R Pars Orbitalis | Grey level co-occurrence | 0.002109 |
| | matrix cluster tendency | |
| | with LHL filter | |
| R Pericalcarine | NGTDM contrast | −1.33727 |
| R Precentral | GLRLM long run high | −0.2258 |
| | grey level emphasis | |
| | with HHL filter | |
| R Rostral | fractal dimension | 1.905555 |
| anterior cingulate | standard deviation | |
| R Superior | First Order Statistics | −1.99215 |
| parietal | minimum with HLL filter | |
| R Transverse | Fractal dimension | −0.90794 |
| Temporal | lacunarity with | |
| | LLH filter | |
| R Transverse | First order | 0.634112 |
| Temporal | statistics range | |

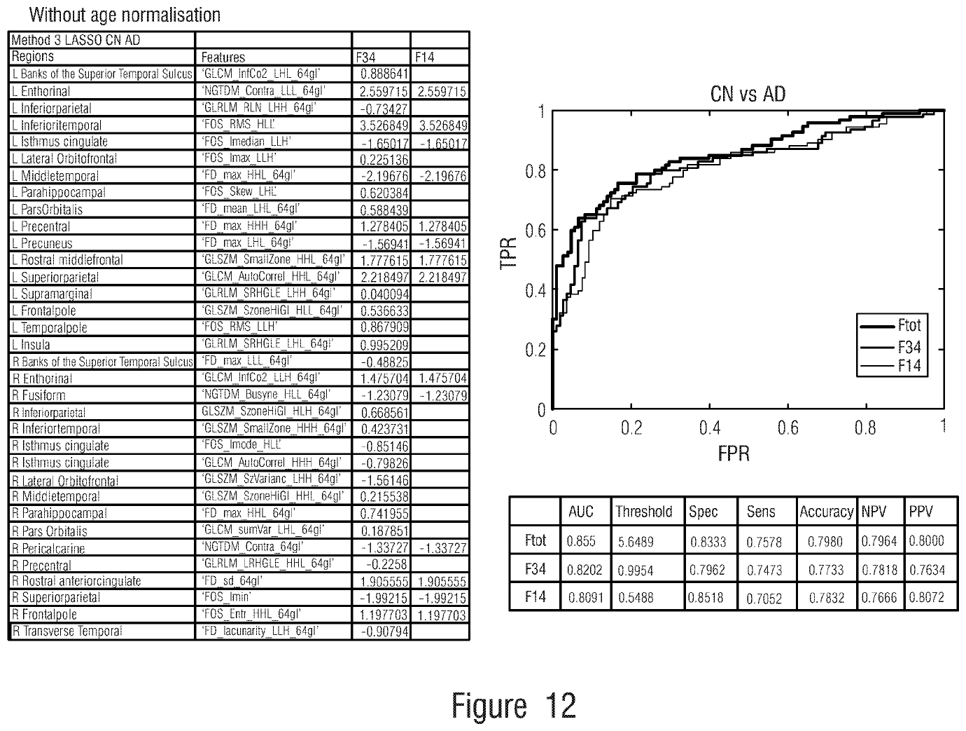

Table 12 comprises 78 tuples in 34 different sub-regions of the cortex. As with the data listed in Table 11 in the comparative test, the highest weighted tuple in each region was selected an used to provide a reduced feature set, labelled F34 in FIG. 12. A further reduced feature set was also constructed by using the most heavily weighted 14 tuples from amongst these 34, labelled F14 in FIG. 12. Again, it can be seen that the reduced feature sets both provide effective diagnostic/predictive power. Again the entorhinal cortex plays a significant role, although in this predictor the NGTDM contrast, a texture feature of the entorhinal is significant.

The consistent finding of the predictors investigated herein is that it is possible to define texture and morphology based models of certain brain regions which can be used to predict the presence of cognitive impairment and/or Alzheimer's disease. A wide variety of models/predictors are possible. Without wishing to be bound by theory, it is believed that those which take account of at least the texture and/or morphology of the entorhinal cortex, the fusiform gyrus, the temporal pole, and the transverse temporal cortex may be the most effective predictors. The insula may also make a significant contribution.

In further investigation to establish the robustness of the methods described and claimed herein a further dataset was investigated in which the control subjects comprised both healthy controls and subjects with Parkinson's and frontotemporal disease. The other subjects, referred to herein as group ADrp, had prodromal Alzheimer's disease and Alzheimer's disease. Predictors were developed that, in a first stage classification were able to distinguish group ADrp from the control subjects. A further predictor was also developed that is able to distinguish within group ADrp. These two predictors differ from those described above, not only in that the control subjects comprise subjects with Parkinson's and frontotemporal disease, but also in that the predictor uses both cortical regions and sub-regions of the cortex.

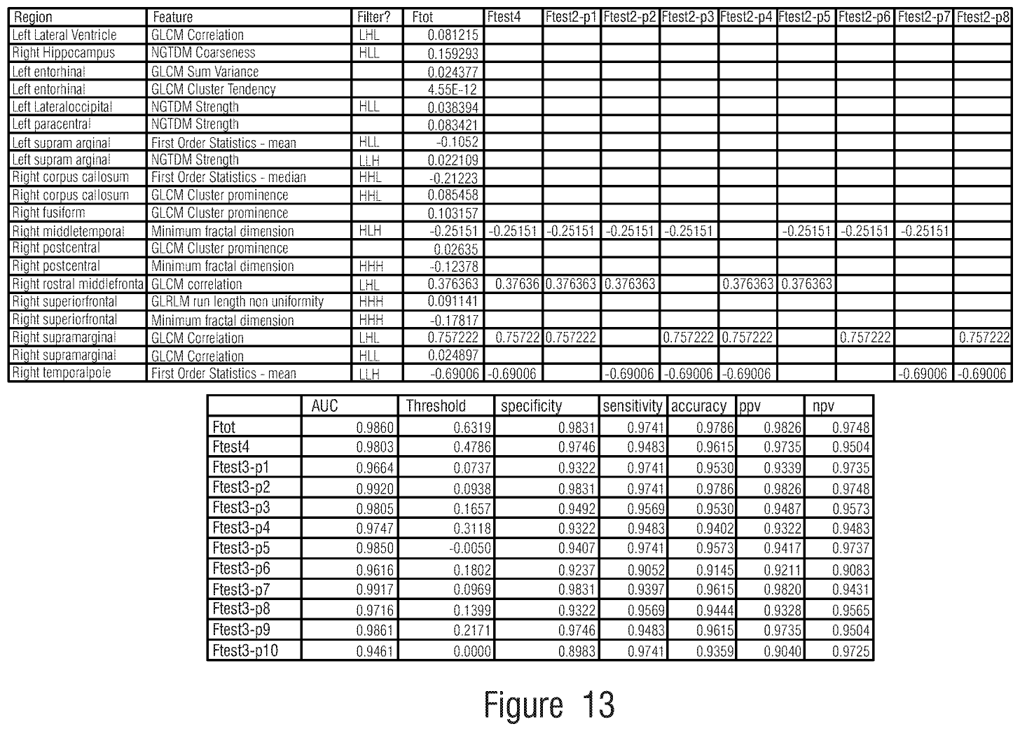

FIG. 13, and Table 13 below set out a predictor to distinguish between (a) control subjects comprising both healthy controls and subjects with Parkinson's and frontotemporal disease, and (b) a group ADrp having both prodromal Alzheimer's disease and Alzheimer's disease.

TABLE 13

| Region | Feature | Filter | Ftot |
|---|---|---|---|
| Left Lateral | GLCM Correlation | LHL | 0.081215 |
| Ventricle | | | |
| Right Hippocampus | NGTDM Coarseness | HLL | 0.159293 |
| Left entorhinal | GLCM Sum Variance | | 0.024377 |
| Left entorhinal | GLCM Cluster Tendency | | 4.55E−12 |
| Left lateraloccipital | NGTDM Strength | HLL | 0.038394 |
| Left paracentral | NGTDM Strength | | 0.083421 |
| Left supramarginal | First Order Statistics - median | HLL | −0.1052 |
| Left supramarginal | NGTDM Strength | LLH | 0.022109 |
| Right corpuscallosum | First Order Statistics - mean | HLL | −0.21223 |
| Right corpuscallosum | GLCM Cluster prominence | HLL | 0.085458 |
| Right fusiform | GLCM Cluster prominence | | 0.103157 |
| Right middletemporal | Minimum fractal dimension | HLH | −0.25151 |
| Right postcentral | GLCM Cluster prominence | | 0.02635 |
| Right postcentral | Minimum fractal dimension | HHH | −0.12378 |
| Right | GLCM correlation | LHL | 0.376363 |
| rostralmiddlefrontal | | | |
| Right superiorfrontal | GLRLM run length | HHH | 0.091141 |
| | non uniformity | | |
| Right superiorfrontal | Minimum fractal dimension | HHH | −0.17817 |
| Right supramarginal | GLCM correlation | LHL | 0.757222 |
| Right supramarginal | GLCM correlation | HLL | 0.024897 |
| Right temporalpole | First Order Statistics - mean | LLH | −0.69006 |

This predictor based on the above features and weights set out in Table 13 was found to provide an AUC of 0.986 in an ROC analysis, and a specificity of 0.9831. It is thus shown to have very reliable predictive power and specificity—even where the control group comprise subjects having other cognitive impairments. A comparative test was performed to demonstrate the possibility to employ reduced feature sets and to demonstrate that the predictive power is preserved when selected ones of the image regions and image metrics are used in the predictor and other are disregarded. As illustrated in FIG. 13, a reduced feature set was constructed using at least two regions selected from the following four:
Right Middle Temporal
Right Rostral Middle Frontal
Right Supramarginal
Right Temporal Pole

It was found that all of the predictors generated using these regions reliably distinguished the group ARDP subjects from the control group—even where the control subjects comprised both healthy controls and subjects with Parkinson's and frontotemporal disease. The use of spatial filters such as those indicated in column 13 of table 13 is optional.

It can be seen that when reduced to three image region-feature tuples, the best performance was given by the predictor labelled permutation 2 (Ftest3-p2) in FIG. 13. This involves Right Middle Temporal, Right Rostral Middle Frontal and Right Temporal Pole. When reduced to 2 Features, best performance given by permutation 7 (Ftest2-p7), which involves Right Middle Temporal and Right Temporal Pole.

In the Right Middle Temporal, this embodiment may employ a measure of complexity such as fractal dimension (e.g. minimum fractal dimension). In the right temporal pole a measure of central tendency of pixel intensity may be used, such as the mean intensity.

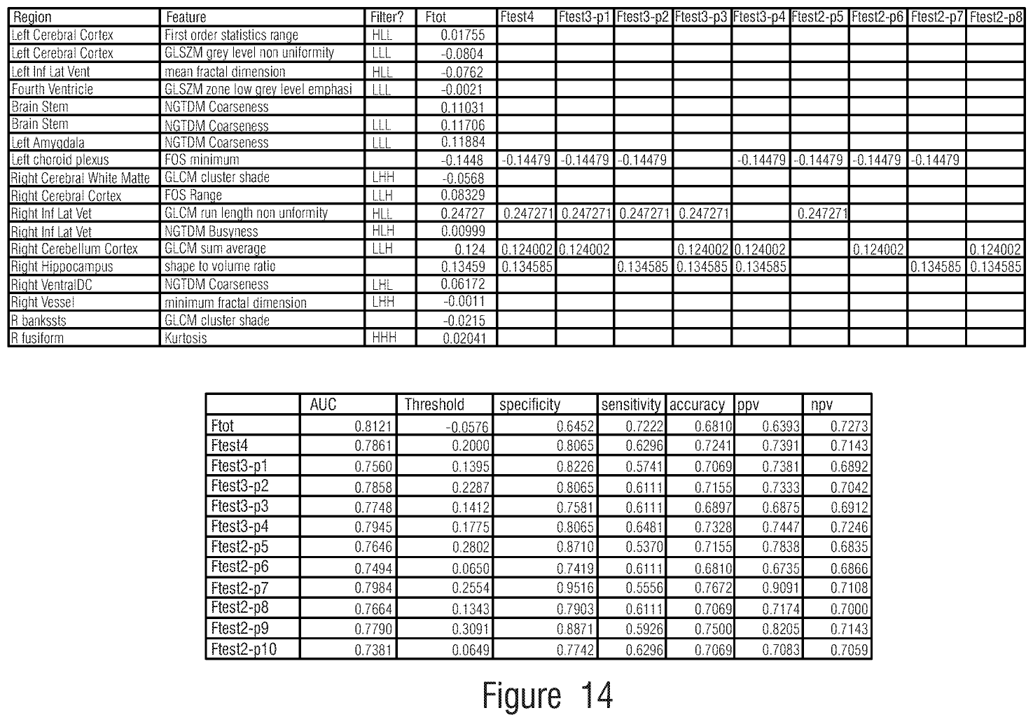

FIG. 14, and Table 14 below set out the results obtained from this approach when the predictor is developed to distinguish between patients having prodromal Alzheimer's disease and those having Alzheimer's disease

TABLE 14

| Region | Feature | Filter | Weight |
|---|---|---|---|
| Left Cerebral | First order statistics range | HLL | 0.01755 |
| Cortex | | | |
| Left Cerebral | GLSZM grey level | LLL | −0.0804 |
| Cortex | non uniformity | | |
| Left Inferior | mean fractal dimension | HLL | −0.0762 |
| Lateral Ventricle | | | |
| Fourth Ventricle | GLSZM zone low grey | LLL | −0.0021 |
| | level emphasis | | |
| Brain Stem | NGTDM Coarseness | | 0.11031 |
| Brain Stem | NGTDM Coarseness | LLL | 0.11706 |
| Left Amygdala | NGTDM Coarseness | LLL | 0.11844 |
| Left choroid | FOS minimum | | −0.1448 |
| plexus | | | |
| Right Cerebral | GLCM cluster shade | LHH | −0.0568 |
| White Matter | | | |
| Right Cerebral | FOS Range | LLH | 0.08329 |
| Cortex | | | |
| Right Inferior | GLRLM run length non | HLL | 0.24727 |
| Lateral Ventricle | unformity | | |
| Right Inferior | NGTDM Busyness | HLH | 0.00999 |
| Lateral Ventricle | | | |
| Right Cerebellar | GLCM sum average | LLH | 0.124 |
| cortex | | | |
| Right Hippocampus | shape to volume ratio | | 0.13459 |
| Right Ventral DC | NGTDM Coarseness | LLL | 0.06172 |
| Right Vessel | minimum fractal dimension | LHH | −0.0011 |
| Right banks of the | GLCM cluster shade | | −0.0215 |
| superior | | | |
| temporal sulcus | | | |
| R fusiform | Kurtosis | HHH | 0.02041 |

This predictor based on the above features and weights set out in Table 14 was found to provide an AUC of 0.8121 in an ROC analysis, and a specificity of 0.65. It is thus shown to have reasonable predictive power and specificity—even where distinguishing between different Alzheimer's disease states. A comparative test was performed to demonstrate the possibility to employ reduced feature sets and to demonstrate that the predictive power is preserved when selected ones of the image regions and image metrics are used in the predictor and other are disregarded. As illustrated in FIG. 14, a reduced feature set was constructed using at least two regions selected from the following four:
Left choroid Plexus
Right Inferior Lateral Ventricle
Right Cerebellar cortex
Right Hippocampus

Surprisingly in this predictor higher accuracy may be achieved by the reduced feature sets. When reduced to these 4 regions & features, the accuracy of the classification goes to 0.72 (compared to 0.68 given by the complete set of features & regions). When reduced to 3 Features, best performance is given by permutation 4 (labelled Ftest3-p4 in FIG. 14), which involves Left choroid Plexus, Right Cerebellar cortex and the Right Hippocampus.

When reduced to 2 Features, the higher accuracy is obtained with permutation 7 (Ftest2-p7), which involves Left choroid Plexus and Right Hippocampus. The feature used in the left choroid plexus may comprise a measure of intensity such as the minimum intensity. The feature used in the right hippocampus may comprise a morphological measure, such as the shape to volume ratio. This permutation gives the highest accuracy.

Embodiments of the disclosure may employ these methods sequentially—in a first classification step, a predictor such as any one of those described above with reference to FIG. 13 and/or Table 13 may be used to diagnose or to predict a cognitive impairment state corresponding to Alzheimer's disease. Then, in the event that this first method step does indicate a cognitive impairment state corresponding to Alzheimer's disease, a predictor such as any one of those described above with reference to FIG. 14 and/or Table 14 may be applied to diagnose or to distinguish between Alzheimer's disease and prodromal Alzheimer's disease in the patient. It will be appreciated in the context of the present disclosure that the prodromal stage of Alzheimer's disease may also referred to as mild cognitive impairment (MCI) due to Alzheimer's disease, and this is the stage where there are obvious symptoms of brain dysfunction. Accordingly, in the present disclosure MCI may comprise the prodromal stage of Alzheimer's disease.

These and other methods of the disclosure may be used to stratify patients for clinical trials and/or to assess the effectiveness of treatments or therapies applied to said patients. It will be appreciated in the context of the present disclosure that whilst particular metrics of image structure in the image regions identified herein have been found to have particular differentiating power, other metrics—e.g. such as metrics of structure, shape, complexity and texture may also be used.

The reference data store used to store data against which test values are compared may comprise volatile and/or non-volatile data storage for storing the above described reference data. The reference data may comprise data calculated by applying the image metrics described herein to a set of reference images of the human brain (a training data set). This training data set may comprise a large number of images of different subjects for which the cognitive impairment diagnosis of each subject is known—e.g. having been verified by other means.

It will be appreciated from the discussion above that the embodiments shown in the Figures are merely exemplary, and include features which may be generalised, removed or replaced as described herein and as set out in the claims. With reference to the drawings in general, it will be appreciated that schematic functional block diagrams are used to indicate functionality of systems and apparatus described herein. It will be appreciated however that the functionality need not be divided in this way, and should not be taken to imply any particular structure of hardware other than that described and claimed below. The function of one or more of the elements shown in the drawings may be further subdivided, and/or distributed throughout apparatus of the disclosure. In some embodiments the function of one or more elements shown in the drawings may be integrated into a single functional unit.

In some examples the functionality of the controller may be provided by a general purpose processor, which may be configured to perform a method according to any one of those described herein. In some examples the controller may comprise digital logic, such as field programmable gate arrays, FPGA, application specific integrated circuits, ASIC, a digital signal processor, DSP, or by any other appropriate hardware. In some examples, one or more memory elements can store data and/or program instructions used to implement the operations described herein. Embodiments of the disclosure provide tangible, non-transitory storage media comprising program instructions operable to program a processor to perform any one or more of the methods described and/or claimed herein and/or to provide data processing apparatus as described and/or claimed herein. The controller may comprise an analogue control circuit which provides at least a part of this control functionality. An embodiment provides an analogue control circuit configured to perform any one or more of the methods described herein.

The embodiments described herein need not perform any diagnosis in order to provide a technical advantage. In particular, there is no need for any comparison with standard data because patients can simply be stratified according to the indicators obtained from the combinations of features (image metrics), image regions, and weights described herein. Such indicators may be used to stratify subjects, for example to identify cohorts for clinical trials Accordingly, methods of the present disclosure comprise computer implemented methods of processing images, such as T1 weighted MRI images to determine any one or more of the indicators based on image regions and image metrics described herein. Such methods may further comprise conducting clinical trials and/or processing clinical measurement data obtained from the subjects to investigate the efficacy of therapies, such as drug treatments. It will thus be appreciated that the methods and apparatus described herein offer a new method of physiological measurement.

The above embodiments are to be understood as illustrative examples. Further embodiments are envisaged. It is to be understood that any feature described in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

1-117. (canceled)

118. A computer implemented method of predicting or diagnosing cognitive impairment in a human subject based on images of the subject's brain, the method comprising:
determining, based on processing by the computer of digital image data obtained from the images, image metrics comprising at least one of:
a complexity metric of an image region corresponding to the right middle temporal gyms;
an image texture metric of a image region corresponding to the right rostral middle frontal;
an image texture metric in the image region corresponding to the right supramarginal; and
an image intensity metric in the image region corresponding to the right temporal pole;
the method further comprising:
determining an indicator based on said image metrics according to a predetermined method; and
predicting or diagnosing cognitive impairment state in the subject based on the indicator.

119. The method of claim 118 wherein predicting or diagnosing cognitive impairment state comprises distinguishing between:
(a) Alzheimer's disease; and
(b) non-Alzheimer's disease.

120. The computer implemented method of claim 118 wherein the image metrics comprise the complexity metric of the image region corresponding to the right middle temporal gyms; and
the image texture metric of the image region corresponding to the right rostral middle frontal.

121. The computer implemented method of claim 120 wherein the image metrics comprise the image texture metric in the image region corresponding to the right supramarginal.

122. The computer implemented method of claim 120 wherein the image metrics comprise the image intensity metric in the image region corresponding to the right temporal pole.

123. The computer implemented method of claim 118 wherein the image metrics comprise the complexity metric of the image region corresponding to the right middle temporal gyms; and the image texture metric in the image region corresponding to the right supramarginal.

124. The computer implemented method of claim 123 wherein the image metrics comprise the image intensity metric in the image region corresponding to the right temporal pole.

125. The computer implemented method of claim 118 wherein the image metrics comprise the image texture metric of the image region corresponding to the right rostral middle frontal; and the image intensity metric in the image region corresponding to the right temporal pole.

126. The computer implemented method of claim 118 wherein the image metrics comprise the image texture metric of the image region corresponding to the right rostral middle frontal; and the image texture metric in the image region corresponding to the right supramarginal.

127. The computer implemented method of claim 126 wherein the image metrics comprise the image intensity metric in the image region corresponding to the right temporal pole.

128. The computer implemented method of claim 118 wherein the image metrics comprise the image texture metric in the image region corresponding to the right supramarginal and the image intensity metric in the image region corresponding to the right temporal pole.

129. The computer implemented method of claim 118 wherein the image metrics comprise the complexity metric of the image region corresponding to the right middle temporal gyms; and the image metrics comprise the image intensity metric in the image region corresponding to the right temporal pole.

130. The computer implemented method of claim 118 wherein the complexity metric of the right middle temporal gyms comprises a measure of fractal dimension such as a minimum fractal dimension, optionally wherein the image data in the region corresponding to the right inferior lateral ventricle is modified using an HLH filter.

131. The computer implemented method of claim 118 wherein the image texture metric in the right supramarginal comprises a measure of correlation, such as the GLCM correlation.

132. The computer implemented method of claim 118 wherein the image texture metric the right rostral middle frontal comprises a measure of correlation, such as the GLCM correlation.

133. The computer implemented method of claim 118 wherein the image intensity metric in the right temporal pole comprises a measure of central tendency such as the mean.

134. The method of claim 118 wherein the predetermined method comprises computing a weighted sum of the image metrics.

135. The method of claim 118 comprising obtaining reference data configured to indicate a cognitive impairment state using reference indicators determined according to the predetermined method, and comparing the indicators for the subject to the reference indicators to perform said predicting or diagnosing of the cognitive impairment state in the subject.

136. (canceled)

137. The computer implemented method of claim 118 comprising operating a processor to automatically segment the images to provide digital image data corresponding to ROIs in each of the image regions, and determining the image metrics by operating the processor to perform, on the digital data, image processing steps configured to provide said image metrics.

138. A computer program product or computer apparatus configured to perform the method of claim 118, and to provide an output indicating said prediction or diagnosis.
